(19) United States
(12) Patent Application Publication
(10) Pub. No.: US 2020/0205734 A1
(43) Pub. Date: Jul. 2, 2020

(54) Ear-Based Physiological State Monitoring

(71) Applicant: Flashback Technologies, Inc., Louisville, CO (US)

(72) Inventors: Isobel Jane Mulligan, Niwot, CO (US); Gregory Zlatko Grudic, Niwot, CO (US); Abhishek Jaiantilal, Highlands Ranch, CO (US)

(21) Appl. No.: 16/726,337

(22) Filed: Dec. 24, 2019

Related U.S. Application Data
(60) Provisional application No. 62/785,113, filed on Dec. 26, 2018.

(51) Int. Cl. A61B 5/00; A61B 5/024

(52) U.S. Cl. CPC A61B 5/6817; A61B 5/0008; A61B 5/742; A61B 5/02416; A61B 5/0022

(57) ABSTRACT

Novel tools and techniques are provided for ear-based physiological data monitoring. A method includes monitoring, with one or more first sensors disposed in a sensor device that is positioned at least partially within an ear canal of a user, physiological data of the user, and sending, with the one or more first sensors and to a computing system, data regarding the monitored physiological data of the user as obtained from within the ear canal of the user. The method further includes analyzing, with the computing system, the received data regarding the monitored physiological data of the user as obtained from within the ear canal of the user, and determining, with the computing system, at least one physiological state of the user, based at least in part on the analysis of the data regarding the monitored physiological data of the user as obtained from within the ear canal of the user.

### CROSS-REFERENCES TO RELATED APPLICATIONS

This application claims priority to U.S. Provisional Patent Application Ser. No. 62/785,113, filed Dec. 26, 2018 by Isobel Jane Mulligan et al. (attorney docket no. 0463.20PR), entitled “Method and System for Implementing Physiological State Measurement of a User Based on Ear-Based Physiologic Data Monitoring,” the entire disclosure of which is incorporated herein by reference in its entirety for all purposes.

This application may be related to U.S. patent application Ser. No. 15/620,701, filed Jun. 12, 2017 by Mulligan et al. and entitled “Rapid Detection of Bleeding Following Injury” (attorney docket no. 0463.17, referred to herein as the “'701 Application”), which claims priority to provisional U.S. Patent Application No. 62/349,516, filed Jun. 13, 2016 by Mulligan et al. and entitled “Rapid Detection of Bleeding Following Injury” (attorney docket no. 0463.17PR, referred to herein as the “'516 Application”), each of which is incorporated herein by reference in its entirety. This application may also be related to U.S. patent application Ser. No. 15/261,661, filed Sep. 9, 2016 by Mulligan et al. and entitled “Estimating Physiological States Based on Changes in CRI” (attorney docket no. 0463.16, referred to herein as the “'661 Application”), which claims priority to the '516 Application and to provisional U.S. Patent Application No. 62/216,187, filed Sep. 9, 2015 by Mulligan et al. and entitled “Estimating Physiological States Based on Changes in CRI” (attorney docket no. 0463.16PR, referred to herein as the “'187 Application”), each of which is incorporated herein by reference in its entirety.

This application may also be related to U.S. patent application Ser. No. 14/885,891, filed Oct. 16, 2015 by Mulligan et al. and entitled “Assessing Effectiveness of CPR” (attorney docket no. 0463.15, referred to herein as the “'891 Application”) and U.S. patent application Ser. No. 14/885,888, filed Oct. 16, 2015 by Mulligan et al. and entitled “Rapid Detection of Bleeding Before, During, and After Fluid Resuscitation” (attorney docket no. 0463.14, referred to herein as the “'888 Application”), each of which claims priority to provisional U.S. Patent Application No. 62/064,816, filed Oct. 16, 2014 by Mulligan et al. and titled “Assessing the Effectiveness of CPR” (attorney docket no. 0463.15PR, referred to herein as the “'816 Application”) and provisional U.S. Patent Application No. 62/064,809 filed Oct. 16, 2014 by Mulligan et al. and titled “Rapid Detection of Bleeding During Fluid Resuscitation” (attorney docket no. 0463.14PR, referred to herein as the “'809 Application”), each of which is incorporated herein by reference in its entirety.

This application may also be related to U.S. patent application Ser. No. 14/542,426, filed Nov. 14, 2014 by Mulligan et al. and titled, “Noninvasive Hydration Monitoring” (attorney docket no. 0463.12, referred to herein as the “'426 Application”) and U.S. patent application Ser. No. 14/542,423, filed Nov. 14, 2014 by Mulligan et al. and titled, “Noninvasive Monitoring for Fluid Resuscitation” (attorney docket no. 0463.11, referred to herein as the “'423 Application”), each of which claims priority to provisional U.S. Patent Application No. 61/905,727, filed Nov. 18, 2013 by Mulligan et al. and titled “Noninvasive Hydration Monitoring” (attorney docket no. 0463.12PR, referred to herein as the “'727 Application”) and provisional U.S. Patent Application No. 61/904,436, filed Nov. 14, 2013 by Mulligan et al. and titled “Noninvasive Monitoring for Fluid Resuscitation” (attorney docket no. 0463.11PR, referred to herein as the “'436 Application”), each of which is incorporated herein by reference in its entirety.

This application may also be related to U.S. patent application Ser. No. 14/535,171, filed Nov. 6, 2014 by Mulligan et al. and titled “Noninvasive Predictive and/or Estimative Blood Pressure Monitoring” (attorney docket no. 0463.10, referred to herein as the “'171 Application”), which claims priority to the '727 Application, the '436 Application, and provisional U.S. Patent Application No. 61/900,980, filed Nov. 6, 2013 by Mulligan et al. and titled “Noninvasive Predictive and/or Estimative Blood Pressure Monitoring” (attorney docket no. 0463.10PR), each of which is incorporated herein by reference in its entirety.

This application may also be related to U.S. patent application Ser. No. 13/554,483, filed Jul. 20, 2012 by Grudic et al. and titled, “Hemodynamic Reserve Monitor and Hemodialysis Control” (attorney docket no. 0463.05, referred to herein as the “'483 Application”; now issued U.S. Pat. No. 9,757,041), which claims priority to provisional U.S. Patent Application No. 61/510,792, filed Jul. 22, 2011 by Grudic et al. and entitled “Cardiovascular Reserve Monitor” (attorney docket no. 0463.05PR, referred to herein as the “'792 Application”) and provisional U.S. Patent Application No. 61/614,426, filed Mar. 22, 2012 by Grudic et al. and entitled “Hemodynamic Reserve Monitor and Hemodialysis Control” (attorney docket no. 0463.07PR, referred to herein as the “'426 Application”), each of which is hereby incorporated by reference in its entirety.

This application may also be related to U.S. patent application Ser. No. 13/041,006, filed Mar. 4, 2011 by Grudic et al. and entitled “Active Physical Perturbations to Enhance Intelligent Medical Monitoring” (attorney docket no. 0463.04, referred to herein as the “'006 Application”), which claims priority to provisional U.S. Patent Application No. 61/310,583, filed Mar. 4, 2010 by Grudic et al. and titled “Active Physical Perturbations to Enhance Intelligent Medical Monitoring” (attorney docket no. 0463.04PR, referred to herein as the “'583 Application”), each of which is hereby incorporated by reference in its entirety.

This application may also be related to U.S. patent application Ser. No. 13/028,140, filed Feb. 15, 2011 by Grudic et al. and entitled “Statistical, Noninvasive Measurement of Intracranial Pressure” (attorney docket no. 0463.03, referred to herein as the “'140 Application”; now issued U.S. Pat. No. 8,512,260), which claims priority to provisional U.S. Patent Application No. 61/305,110, filed Feb. 16, 2010, by Moulton et al. and titled “Statistical, Noninvasive Method for Predicting Intracranial Pressure” (attorney docket no. 0463.03PR, referred to herein as the “'110 Application”), each of which is hereby incorporated by reference in its entirety.

This application may also be related to International Application No. PCT/US2009/062119, filed Oct. 26, 2009 by Grudic et al. and entitled “Long Term Active Learning from Large Continually Changing Data Sets” (attorney docket no. 0463.01/PCT, referred to herein as the “'119 Application”), which claims priority to provisional U.S. Patent Application No. 61/252,978, filed Oct. 19, 2009 by Grudic et al. and titled “Long Term Active Learning from Large Continually Changing Data Sets,” provisional U.S. Patent Application No. 61/166,499, filed Apr. 3, 2009 by Moulton and titled “Advances in Pre-Hospital Care,” provisional U.S. Patent Application No. 61/166,486, filed Apr. 3, 2009 by Grudic et al. and titled “Statistical Methods for Predicting Patient Specific Blood Loss Volume Causing Hemodynamic Decompensation,” provisional U.S. Patent Application No. 61/166,472, filed Apr. 3, 2009 by Grudic et al. and titled “Long Term Active Learning from Large Continually Changing Data Sets,” and provisional U.S. Patent Application No. 61/109,490, filed Oct. 29, 2008 by Moulton et al. and titled “Method for Determining Physiological State or Condition,” each of which is hereby incorporated by reference in its entirety.

The respective disclosures of these applications/patents (which this document refers to collectively as the “Related Applications”) are incorporated herein by reference in their entirety for all purposes.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

### FIELD

The present disclosure relates, in general, to methods, systems, and apparatuses for implementing physiological monitoring, and, more particularly, to methods, systems, and apparatuses for implementing physiological state measurement of a user based on ear-based physiological data monitoring.

### BACKGROUND

With the introduction of portable personal sensors in wearable devices that track or monitor certain physiological characteristics of a person (e.g., pulse or heart rate, step count, etc.), individuals can now roughly gauge their estimated health. Such estimates, however, are extremely rough, and not precise, owing to other unmeasured factors that may break any causal relationships between the pulse or heart rate and/or step count (or other monitored physiological characteristics of an individual and the individual's health.

In particular, most commercially available personal trackers or the like are incapable of accurately determining a compensator reserve and/or a heart rate of an individual based on thermal or pulsatile waveform monitoring of an ear canal of the individual.

Hence, there is a need for more robust and scalable solutions for implementing physiological monitoring, and, more particularly, to methods, systems, and apparatuses for implementing physiological state measurement of a user based on ear-based physiological data monitoring.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. In some instances, a sub-label is associated with a reference numeral to denote one of multiple similar components. When reference is made to a reference numeral without specification to an existing sub-label, it is intended to refer to all such multiple similar components.

FIG. 1 is a schematic diagram illustrating a system for implementing physiological state measurement of a user based on ear-based physiological data monitoring, in accordance with various embodiments.

FIG. 2 is a schematic diagram illustrating a system for estimating compensatory reserve, which can be used for implementing physiological state measurement of a user based on ear-based physiological data monitoring, in accordance with various embodiments.

FIG. 3 is a schematic diagram illustrating an example of a sensor system that can be placed in an ear canal of a patient and that can be used for implementing physiological state measurement of a user based on ear-based physiological data monitoring, in accordance with various embodiments.

FIGS. 4A-4C are flow diagrams illustrating a method for implementing physiological state measurement of a user based on ear-based physiological data monitoring, in accordance with various embodiments.

FIG. 5 is a block diagram illustrating an exemplary computer or system hardware architecture, in accordance with various embodiments.

### DETAILED DESCRIPTION OF CERTAIN EMBODIMENTS

Overview

Various embodiments provide tools and techniques for implementing physiological monitoring, and, more particularly, to methods, systems, and apparatuses for implementing physiological state measurement of a user based on ear-based physiological data monitoring.

In various embodiments, one or more first sensors—which might be disposed in sensor device(s) that is at least partially displaced or inserted within an ear canal of a user—might monitor physiological data of the user, and might send data regarding the monitored physiological data of the user as obtained from within the ear canal of the user to a computing system. The computing system might receive the data regarding the monitored physiological data of the user as obtained from within the ear canal of the user; might analyze the received data regarding the monitored physiological data of the user as obtained from within the ear canal of the user; might determine at least one physiological state of the user, based at least in part on the analysis of the data regarding the monitored physiological data of the user as obtained from within the ear canal of the user; and might send the determined at least one physiological state of the user to the user device(s) of the user for display on a display screen of the user device(s).

In some embodiments, monitoring the physiological data of the user might comprise monitoring a thermal reading from within the ear canal of the user. In such cases, determining the at least one physiological state of the user might comprise determining, with the computing system, a body temperature of the user, based at least in part on the monitored thermal reading.

Alternatively, or additionally, monitoring the physiological data of the user might comprise monitoring a pulsatile waveform that is detected in the ear canal of the user. In such cases, determining the at least one physiological state of the user might comprise calculating, with the computing system, at least one of a compensatory reserve index (“CRI”) or a heart rate (“HR”) of the user based at least in part on the monitored pulsatile waveform.

Merely by way of example, according to some embodiments, displaying the determined at least one physiological state of the user might comprise at least one of: displaying, on the display screen of the user device, an indication that the sensor device has been properly placed within the ear canal to sufficiently capture sensor readings; displaying, on the display screen of the user device, an indication that the sensor device has not been properly placed within the ear canal to sufficiently capture sensor readings and a set of instructions to properly insert the sensor device within the ear canal after removal of the sensor device from the ear canal; displaying, on the display screen of the user device, a body temperature of the user as measured by the one or more first sensors from within the ear canal of the user; displaying, on the display screen of the user device, a compensatory reserve index (“CRI”) as calculated based at least in part on pulsatile waveform readings as obtained by the one or more first sensors from within the ear canal of the user; displaying, on the display screen of the user device, a heart rate (“HR”) of the user as measured by the one or more first sensors from within the ear canal of the user; displaying, on the display screen of the user device, at least one notification indicating that one or more of CRI, HR, or body temperature readings are valid and complete; or displaying, on the display screen of the user device, one or more error messages when at least one of an error occurs or no valid readings can be made; and/or the like.

Correspondingly, in some embodiments, the determined at least one physiological state of the user might be audibly presented to the user (e.g., using one or more speakers of user device(s), or the like). In some cases, audibly presenting the determined at least one physiological state of the user might comprise at least one of: audibly presenting, using one or more speakers of the user device, an indication that the sensor device has been properly placed within the ear canal to sufficiently capture sensor readings; audibly presenting, using the one or more speakers of the user device, an indication that the sensor device has not been properly placed within the ear canal to sufficiently capture sensor readings and a set of instructions to properly insert the sensor device within the ear canal after removal of the sensor device from the ear canal; audibly presenting, using the one or more speakers of the user device, a body temperature of the user as measured by the one or more first sensors from within the ear canal of the user; audibly presenting, using the one or more speakers of the user device, a compensatory reserve index (“CRI”) as calculated based at least in part on pulsatile waveform readings as obtained by the one or more first sensors from within the ear canal of the user; audibly presenting, using the one or more speakers of the user device, a heart rate (“HR”) of the user as measured by the one or more first sensors from within the ear canal of the user; audibly presenting, using the one or more speakers of the user device, one or more beeps or tones indicating that one or more of CRI, HR, or body temperature readings are valid and complete; or audibly presenting, using the one or more speakers of the user device, one or more error messages when at least one of an error occurs or no valid readings can be made; and/or the like.

According to some aspects, a measurement of compensatory reserve, as characterized by a compensatory reserve index (which is described in detail in the Related Applications, which have already been incorporated herein by reference in their entirety for all purposes), may be indicative of a body state of the user, as measured from within the ear canal of the user.

In some embodiments, the physiological state determination may be performed in real-time or near-real-time based on the monitored sensor data (i.e., physiological data collected or monitored by the one or more first sensors).

In some aspects, the method and system of the various embodiments might allow for a portable device or system that may at least in part be removably inserted within an ear canal of a user to monitor temperature, pulsatile waveforms, and/or other physiological characteristics of the user. The system might calculate CRI of the user (where measurement of CRI in general has been described in detail in the Related Applications, which have already been incorporated herein by reference in their entirety for all purposes), calculate heart rate (“HR”), or other physiological states of the user. In some cases, the system might provide real-time or near-real-time assessments of the health, fitness, or other physiological states of the user based at least in part on monitored physiological data of the user (and, in some cases, based at least in part on measured CRI of the user), as measured from within the ear canal of the user.

These and other aspects of the physiological state measurement of a user based on ear-based physiological data monitoring are described in greater detail with respect to the figures.

The following detailed description illustrates a few exemplary embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. In other instances, certain structures and devices are shown in block diagram form. Several embodiments are described herein, and while various features are ascribed to different embodiments, it should be appreciated that the features described with respect to one embodiment may be incorporated with other embodiments as well. By the same token, however, no single feature or features of any described embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

Various embodiments described herein, while embodying (in some cases) software products, computer-performed methods, and/or computer systems, represent tangible, concrete improvements to existing technological areas, including, without limitation, personal tracking technology, health monitoring technology, and/or the like. In other aspects, certain embodiments, can improve the functioning of user equipment or systems themselves (e.g., personal trackers, portable health monitors, etc.), for example, by monitoring, with one or more first sensors disposed in a sensor device that is at least partially displaced within an ear canal of a user, physiological data of the user; sending, with the one or more first sensors and to a computing system, data regarding the monitored physiological data of the user as obtained from within the ear canal of the user; receiving, with the computing system, the data regarding the monitored physiological data of the user as obtained from within the ear canal of the user; analyzing, with the computing system, the received data regarding the monitored physiological data of the user as obtained from within the ear canal of the user; determining, with the computing system, at least one physiological state of the user, based at least in part on the analysis of the data regarding the monitored physiological data of the user as obtained from within the ear canal of the user; sending, with the computing system and to a user device, the determined at least one physiological state of the user; and displaying, on a display screen of the user device, the determined at least one physiological state of the user; and/or the like.

In particular, to the extent any abstract concepts are present in the various embodiments, those concepts can be implemented as described herein by devices, software, systems, and methods that involve specific novel functionality (e.g., steps or operations), such as, allowing for a portable device or system to be removably inserted within an ear canal of a user to monitor physiological data of the user from within the ear canal (said physiological data including, without limitation, temperature of the user, pulsatile waveform, etc.) and allowing for the system to calculate CRI and heart rate of the user based on the monitored physiological data as measured from within the ear canal. These physiological states of the user may be provided in real-time or near-real-time assessments of the health, fitness, or other physiological states of the user based at least in part on monitored physiological data of the user (and, in some cases, based at least in part on measured CRI of the user), which produce tangible results outside of the implementing computer system.

In an aspect, a method might comprise monitoring, with one or more first sensors disposed in a sensor device that is at least partially displaced within an ear canal of a user, physiological data of the user; sending, with the one or more first sensors and to a computing system, data regarding the monitored physiological data of the user as obtained from within the ear canal of the user; and receiving, with the computing system, the data regarding the monitored physiological data of the user as obtained from within the ear canal of the user. The method might also comprise analyzing, with the computing system, the received data regarding the monitored physiological data of the user as obtained from within the ear canal of the user; and determining, with the computing system, at least one physiological state of the user, based at least in part on the analysis of the data regarding the monitored physiological data of the user as obtained from within the ear canal of the user. The method might further comprise sending, with the computing system and to a user device, the determined at least one physiological state of the user; and displaying, on a display screen of the user device, the determined at least one physiological state of the user.

In some embodiments, the one or more first sensors might comprise at least one of one or more thermal sensors, one or more optical sensors, or one or more heart rate sensors, and/or the like. In some cases, the one or more first sensors might be encapsulated within a sensor device, wherein the sensor device might comprise an ear-plug-shaped housing configured to fit within the ear canal of the user. In some instances, the user device might comprise one of a smart phone, a mobile phone, a smart watch, a tablet computer, a laptop computer, a desktop computer, or a dedicated sensor control device, and/or the like.

According to some embodiments, monitoring the physiological data of the user might comprise monitoring a thermal reading from within the ear canal of the user, wherein determining the at least one physiological state of the user might comprise determining, with the computing system, a body temperature of the user, based at least in part on the monitored thermal reading. Alternatively, or additionally, monitoring the physiological data of the user might comprise monitoring a pulsatile waveform that is detected in the ear canal of the user, wherein determining the at least one physiological state of the user might comprise calculating, with the computing system, at least one of a compensatory reserve index (“CRI”) or a heart rate (“HR”) of the user based at least in part on the monitored pulsatile waveform.

Merely by way of example, according to some embodiments, displaying the determined at least one physiological state of the user might comprise at least one of: displaying, on the display screen of the user device, an indication that the sensor device has been properly placed within the ear canal to sufficiently capture sensor readings; displaying, on the display screen of the user device, an indication that the sensor device has not been properly placed within the ear canal to sufficiently capture sensor readings and a set of instructions to properly insert the sensor device within the ear canal after removal of the sensor device from the ear canal; displaying, on the display screen of the user device, a body temperature of the user as measured by the one or more first sensors from within the ear canal of the user; displaying, on the display screen of the user device, a compensatory reserve index (“CRI”) as calculated based at least in part on pulsatile waveform readings as obtained by the one or more first sensors from within the ear canal of the user; displaying, on the display screen of the user device, a heart rate (“HR”) of the user as measured by the one or more first sensors from within the ear canal of the user; displaying, on the display screen of the user device, at least one notification indicating that one or more of CRI, HR, or body temperature readings are valid and complete; or displaying, on the display screen of the user device, one or more error messages when at least one of an error occurs or no valid readings can be made; and/or the like.

Correspondingly, in some embodiments, the method might further comprise at least one of: audibly presenting, using one or more speakers of the user device, an indication that the sensor device has been properly placed within the ear canal to sufficiently capture sensor readings; audibly presenting, using the one or more speakers of the user device, an indication that the sensor device has not been properly placed within the ear canal to sufficiently capture sensor readings and a set of instructions to properly insert the sensor device within the ear canal after removal of the sensor device from the ear canal; audibly presenting, using the one or more speakers of the user device, a body temperature of the user as measured by the one or more first sensors from within the ear canal of the user; audibly presenting, using the one or more speakers of the user device, a compensatory reserve index (“CRI”) as calculated based at least in part on pulsatile waveform readings as obtained by the one or more first sensors from within the ear canal of the user; audibly presenting, using the one or more speakers of the user device, a heart rate (“HR”) of the user as measured by the one or more first sensors from within the ear canal of the user; audibly presenting, using the one or more speakers of the user device, one or more beeps or tones indicating that one or more of CRI, HR, or body temperature readings are valid and complete; or audibly presenting, using the one or more speakers of the user device, one or more error messages when at least one of an error occurs or no valid readings can be made; and/or the like.

In another aspect, an apparatus might comprise at least one processor and a non-transitory computer readable medium communicatively coupled to the at least one processor. The non-transitory computer readable medium might have stored thereon computer software comprising a set of instructions that, when executed by the at least one processor, causes the apparatus to: receive, from one or more first sensors disposed in a sensor device that is at least partially displaced within an ear canal of a user, data regarding monitored physiological data of the user as obtained from within the ear canal of the user; analyze the received data regarding the monitored physiological data of the user as obtained from within the ear canal of the user; determine at least one physiological state of the user, based at least in part on the analysis of the data regarding the monitored physiological data of the user as obtained from within the ear canal of the user; send, to a user device, the determined at least one physiological state of the user; and displaying, on a display screen of the user device, the determined at least one physiological state of the user.

In yet another aspect, a system might comprise one or more first sensors and a computing system. The one or more first sensors might be disposed in a sensor device that is at least partially displaced within an ear canal of a user, wherein the one or more first sensors might be used to monitor physiological data of the user. The computing system might comprise at least one first processor and a first non-transitory computer readable medium communicatively coupled to the at least one first processor. The first non-transitory computer readable medium might have stored thereon computer software comprising a first set of instructions that, when executed by the at least one first processor, causes the computing system to: receive, from the one or more first sensors, data regarding monitored physiological data of the user as obtained from within the ear canal of the user; analyze the received data regarding the monitored physiological data of the user as obtained from within the ear canal of the user; determine at least one physiological state of the user, based at least in part on the analysis of the data regarding the monitored physiological data of the user as obtained from within the ear canal of the user; send, to a user device, the determined at least one physiological state of the user; and displaying, on a display screen of the user device, the determined at least one physiological state of the user.

In some embodiments, the one or more first sensors might comprise at least one of one or more thermal sensors, one or more optical sensors, or one or more heart rate sensors, and/or the like. In some cases, the one or more first sensors might be encapsulated within a sensor device, wherein the sensor device might comprise an ear-plug-shaped housing configured to fit within the ear canal of the user. In some instances, the user device might comprise one of a smart phone, a mobile phone, a smart watch, a tablet computer, a laptop computer, a desktop computer, or a dedicated sensor control device, and/or the like.

According to some embodiments, monitoring the physiological data of the user might comprise monitoring a thermal reading from within the ear canal of the user, wherein determining the at least one physiological state of the user might comprise determining a body temperature of the user, based at least in part on the monitored thermal reading. Alternatively, or additionally, monitoring the physiological data of the user might comprise monitoring a pulsatile waveform that is detected in the ear canal of the user, wherein determining the at least one physiological state of the user might comprise calculating at least one of a compensatory reserve index (“CRI”) or a heart rate (“HR”) of the user based at least in part on the monitored pulsatile waveform.

Merely by way of example, according to some embodiments, displaying the determined at least one physiological state of the user might comprise at least one of: displaying, on the display screen of the user device, an indication that the sensor device has been properly placed within the ear canal to sufficiently capture sensor readings; displaying, on the display screen of the user device, an indication that the sensor device has not been properly placed within the ear canal to sufficiently capture sensor readings and a set of instructions to properly insert the sensor device within the ear canal after removal of the sensor device from the ear canal; displaying, on the display screen of the user device, a body temperature of the user as measured by the one or more first sensors from within the ear canal of the user; displaying, on the display screen of the user device, a compensatory reserve index (“CRI”) as calculated based at least in part on pulsatile waveform readings as obtained by the one or more first sensors from within the ear canal of the user; displaying, on the display screen of the user device, a heart rate (“HR”) of the user as measured by the one or more first sensors from within the ear canal of the user; displaying, on the display screen of the user device, at least one notification indicating that one or more of CRI, HR, or body temperature readings are valid and complete; or displaying, on the display screen of the user device, one or more error messages when at least one of an error occurs or no valid readings can be made; and/or the like.

Correspondingly, in some embodiments, the first set of instructions, when executed by the at least one first processor, further causes the computing system to perform at least one of: audibly presenting, using one or more speakers of the user device, an indication that the sensor device has been properly placed within the ear canal to sufficiently capture sensor readings; audibly presenting, using the one or more speakers of the user device, an indication that the sensor device has not been properly placed within the ear canal to sufficiently capture sensor readings and a set of instructions to properly insert the sensor device within the ear canal after removal of the sensor device from the ear canal; audibly presenting, using the one or more speakers of the user device, a body temperature of the user as measured by the one or more first sensors from within the ear canal of the user; audibly presenting, using the one or more speakers of the user device, a compensatory reserve index (“CRI”) as calculated based at least in part on pulsatile waveform readings as obtained by the one or more first sensors from within the ear canal of the user; audibly presenting, using the one or more speakers of the user device, a heart rate (“HR”) of the user as measured by the one or more first sensors from within the ear canal of the user; audibly presenting, using the one or more speakers of the user device, one or more beeps or tones indicating that one or more of CRI, HR, or body temperature readings are valid and complete; or audibly presenting, using the one or more speakers of the user device, one or more error messages when at least one of an error occurs or no valid readings can be made; and/or the like.

Various modifications and additions can be made to the embodiments discussed without departing from the scope of the invention. For example, while the embodiments described above refer to particular features, the scope of this invention also includes embodiments having different combination of features and embodiments that do not include all of the above described features.

Compensatory Reserve Index (“CRI”)

Various embodiments can assess the effectiveness of fluid intake hydration, where effectiveness can be defined as, but not limited to, leading to a better hydration state or maintain an optimal hydration state. In one aspect, optimal hydration might be defined as a fluid state that maximized some performance index/measure, perhaps indicated by the patient's compensatory reserve index (“CRI,” also referred to herein and in the Related Applications as “cardiac reserve index” or “hemodynamic reserve index” (“HDRI”), all of which should be considered synonymous for purposes of this disclosure). (While the term, “patient,” is used herein for convenience, that descriptor should not be considered limiting, because various embodiments can be employed both in a clinical setting and outside any clinical setting, such as by an athlete before, during, or after an athletic contest or training, a person during daily activities, a soldier on the battlefield, etc. Thus, the term, “patient,” as used herein, should be interpreted broadly and should be considered to be synonymous with “person.”) In other cases, the assessments might be based on raw waveform data (e.g., PPG waveform data) captured by a sensor on the patient (such as the sensors described below and the Related Applications, for example). In further cases, a combination of waveform data and calculated/estimated CRI can be used to calculate the effectiveness of hydration and/or the amount of fluid needed for effective hydration. In other aspects, such functionality can be provided by and/or integrated with systems, devices (such as a cardiac reserve monitor and/or wrist-worn sensor device, or the like), tools, techniques, methods, and software described below and in the Related Applications.

For example, one set of embodiments provides methods. An exemplary method might comprise monitoring, with one or more sensors, physiological data of a patient. The method might further comprise analyzing, with a computer system, the physiological data. Many different types of physiological data can be monitored and/or analyzed by various embodiments, including, without limitation, blood pressure waveform data, plethysmograph waveform data, photoplethysmograph (“PPG”) waveform data (such as that generated by a pulse oximeter), and/or the like. In an aspect of some embodiments, analyzing the physiological data might comprise analyzing the data against a pre-existing model. In some cases, the method can further comprise assessing the effectiveness of hydration efforts, and/or displaying (e.g., on a display device) an assessment of the effectiveness of the hydration efforts. Such an assessment can include, without limitation, an estimate of the effectiveness at a current time, a prediction of the effectiveness at some point in the future, an estimate and/or prediction of a volume of fluid necessary for effective hydration, an estimate of the probability a patient requires fluids, etc.

An apparatus, in accordance with yet another set of embodiments, might comprise a computer readable medium having encoded thereon a set of instructions executable by one or more computers to perform one or more operations. In some embodiments, the set of instructions might comprise instructions for performing some or all of the operations of methods provided by certain embodiments.

A system, in accordance with yet another set of embodiments, might comprise one or more processors and a computer readable medium in communication with the one or more processors. The computer readable medium might have encoded thereon a set of instructions executable by the computer system to perform one or more operations, such as the set of instructions described above, to name one example. In some embodiments, the system might further comprise one or more sensors and/or a therapeutic device, either or both of which might be in communication with the processor and/or might be controlled by the processor. Such sensors can include, but are not limited to, a blood pressure sensor, an intracranial pressure monitor, a central venous pressure monitoring catheter, an arterial catheter, an electroencephalograph, a cardiac monitor, a transcranial Doppler sensor, a transthoracic impedance plethysmograph, a pulse oximeter, a near infrared spectrometer, a ventilator, an accelerometer, an electrooculogram, a transcutaneous glucometer, an electrolyte sensor, and/or an electronic stethoscope.

CRI for Determining Body States, Transition Between Body States, and Physiological States of User

The inventors of this application have found that physical activity or a change in physical activity of the user (e.g., a body state (as described herein) or a transition between body states (as also described herein)) may result in an impulse response in the CRI of the user. Profiles of the CRI (as compared with base measurements of CRI of the individual user or a compilation of measurements of CRI across a sample of multiple users) may be indicative of health, fitness, and/or other physiological states of the user. In such cases, a CRI server or other computational device might monitor physiological data of the user (e.g., by using sensors, including, but not limited to the one or more first sensors, as described herein) to measure a CRI of the user, and might further analyze the measured CRI to determine a body state of the user and/or a transition between body states of the user, and might also further analyze the measured CRI to determine the physiological state of the user—including, but not limited to, at least one of a hydration state of the user, a dehydration state of the user, a fitness state of the user, a health state of the user, an exertion readiness state of the user, a fatigue state of the user, an alertness level of the user, an altitude sickness state of the user, a level of tolerance to heat of the user, a level of tolerance to cold of the user, a level of tolerance to other environmental conditions of the user, a level of tolerance to liquid limitations of the user, a level of tolerance to blood loss of the user, or one or more states of illness of the user (including, but not limited to, flu, cold, viral infection, bacterial infection, sepsis, heart disease, and/or the like), and/or the like. Such physiological states may then be presented to the user (or a physician or other healthcare provider of the user) using a user interface of a user device, a display screen of a user device, a web portal, a software application (“app”), and/or the like. These functionalities are described in detail in the 0463.18PR Application.

CRI for Assessing Blood Loss

A set of embodiments provides methods, systems, and software that can be used, in many cases noninvasively, to quickly and accurately assess blood loss in a patient (e.g., before, during, and/or after fluid resuscitation). Such an assessment can include, without limitation, an estimate of the effectiveness at a current time, a prediction of the effectiveness at some point in the future, an estimate and/or prediction of a volume of fluid necessary for effective hydration, an estimate of the probability a patient requires fluids, an estimate and/or prediction of blood loss (e.g., before, during, and/or after fluid resuscitation), etc. In a particular set of embodiments, a device, which can be worn on the patient's body, can include one or more sensors that monitor a patient's physiological parameters. The device (or a computer in communication with the device) can analyze the data captured by the sensors and compare such data with a model (which can be generated in accordance with other embodiments) to assess the effectiveness of hydration, as described in further detail in the '426 Application, and/or to assess blood loss (e.g., before, during, and/or after fluid resuscitation).

Different embodiments can measure a number of different physiological parameters from the patient, and the analysis of those parameters can vary according to which parameters are measured (and which, according to the generated model, are found to be most predictive of the effectiveness of hydration, including the probability of the need for hydration and/or the volume of fluids needed, or most predictive of blood loss). In some cases, the parameters themselves (e.g., continuous waveform data captured by a photoplethysmograph) can be analyzed against the model to make assessments of hydration effectiveness or assessments of blood loss (e.g., before, during, and/or after fluid resuscitation). In other cases, physiological parameters can be derived from the captured data, and these parameters can be used Merely by way of example, as described further below and the '483 Application (already incorporated by reference), direct physiological data (captured by sensors) can be used to estimate a value of CRI, and this value of CRI can be used to assess the effectiveness of hydration and/or to assess blood loss (e.g., before, during, and/or after fluid resuscitation). In yet other cases, the derived CRI values and raw sensor data can be used together to perform such assessments.

For example, the '483 Application describes a compensatory reserve monitor (also described as a cardiac reserve monitor or hemodynamic reserve monitor) that is able to estimate the compensatory reserve of a patient. In an aspect, this monitor quickly, accurately, and/or in real-time can determine the probability of whether a patient is bleeding. In another aspect, the device can simultaneously monitor the patient's compensatory reserve by tracking the patient's CRI, to appropriately and effectively guide hydration and ongoing patient care. The same device (or a similar device) can also include advanced functionality to assess the effectiveness of hydration, based on the monitored CRI values, as explained in further detail in the '426 Application, and/or to rapidly assess blood loss (e.g., before, during, and/or after fluid resuscitation).

CRI is a hemodynamic parameter that is indicative of the individual-specific proportion of intravascular fluid reserve remaining before the onset of hemodynamic decompensation. CRI has values that range from 1 to 0, where values near 1 are associated with normovolemia (normal circulatory volume) and values near 0 are associated with the individual specific circulatory volume at which hemodynamic decompensation occurs.

The mathematical formula of CRI, at some time “t” is given by the following equation:

$$C R I ( t ) = 1 - \frac{B L V ( t )}{B L {V}_{H D D}} \qquad ( Eq . 1 )$$

where BLV(t) is the intravascular volume loss (“BLV,” also referred to as “blood loss volume” in the Related Applications) of a person at time “t,” and BLV_{HDD} is the intravascular volume loss of a person when they enter hemodynamic decompensation (“HDD”). Hemodynamic decompensation is generally defined as occurring when the systolic blood pressure falls below 70 mmHg. This level of intravascular volume loss is individual specific and will vary from subject to subject.

Lower body negative pressure (“LBNP”) in some linear or nonlinear relationship λ with intravascular volume loss:

BLV=λ·LBNP (Eq. 2)

can be used in order to estimate the CRI for an individual undergoing a LBNP experiment as follows:

$$CRI = 1 - \frac{B L V ( L )}{B L {V}_{H D D}} \approx 1 - \frac{λ \cdot LBNP ( t )}{λ \cdot {LBNP}_{H D D}} = 1 - \frac{L B NP ( t )}{L B N {P}_{H D D}} \qquad ( Eq . 3 )$$

where LBNP(t) is the LBNP level that the individual is experiencing at time “t,” and, LBNP_{HDD} is the LNPB level that the individual will enter hemodynamic decompensation.

Using either CRI data, raw (or otherwise processed) sensor data, or both, various embodiments can assess the effectiveness of hydration. In one embodiment, the assessment of blood loss (“BL”) can be expressed as a value between 0 and 1; when BL=1, blood loss is certain, when BL=0, there is no blood loss, and when BL is a value between 1 and 0, the value is indicative of probability of blood loss, perhaps due to ongoing bleeding before, during, and/or after fluid resuscitation. (Of course, other embodiments can scale the value of BL differently). In an aspect of some embodiments, a general expression for the estimate of blood loss is as follows:

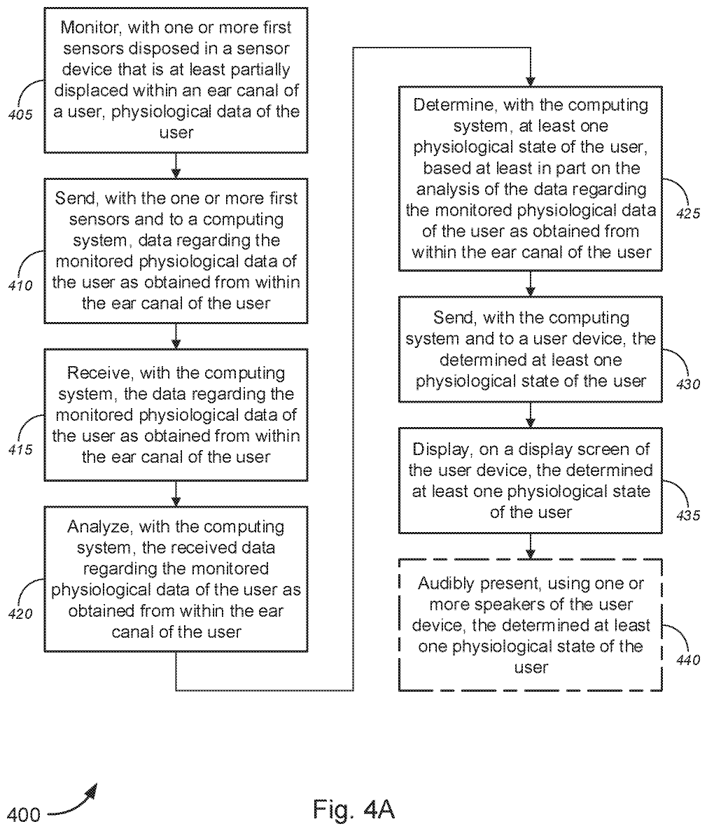

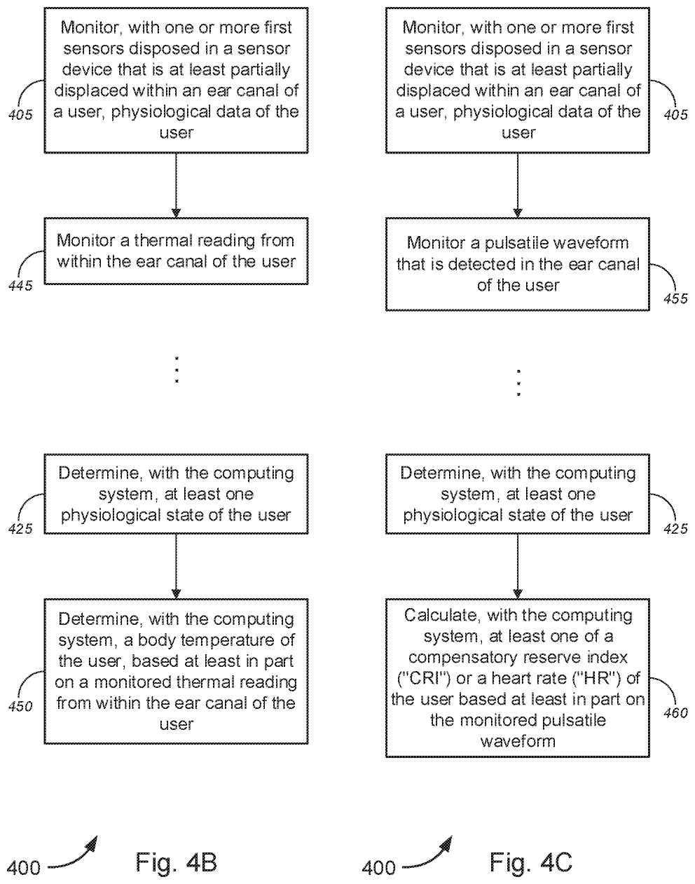

BL=ƒ_{BL}(CRIₜ,FVₜ,Sₜ) (Eq. 4)

where BL is a measure or an estimate of blood loss, ƒ_{BL}(CRIₜ, FVₜ, Sₜ) is an algorithm embodied by a model generated empirically, e.g., using the techniques described with respect to FIG. 4 below, and/or in the Related Applications, CRIₜ is a time history of CRI values (which can range from a single CRI value to many hours of CRI values), FVₜ is a time history of fluid volume being given to the patient (which can range from a single value to many hours of values), and Sₜ is a time history of raw sensor values, such as physiological data measured by the sensors, as described elsewhere herein (which can range from one value to many hours of values).

The functional form of Eq. 4 is similar to but not limited to the form of the CRI model in the sense that time histories of (CRIₜ, FVₜ, Sₜ) data gathered from human subjects at various levels of BL are compared to time histories of (CRIₜ, FVₜ, Sₜ) for the current patient being monitored. The estimated BL for the current patient is then that which is the closest in (CRIₜ, FVₜ, Sₜ) space to the previously gathered data.

While Eq. 4 is the general expression for BL, various embodiments might use subsets of the parameters considered in Eq. 4. For instance, in one embodiment, a model might consider only the volume of fluid and CRI data, without accounting for raw sensor input. In that case, BL can be calculated as follows:

BL=ƒ_{BL}(CRIₜ,FVₜ). (Eq. 5)

Similarly, some models might estimate BL based on sensor data, rather than first estimating CRI, in which case, BL can be expressed thusly:

BL=ƒ_{BL}(FVₜ,Sₜ). (Eq. 6)

The choice of parameters to use in modeling BL is discretionary, and it can depend on what parameters are shown (e.g., using the techniques of FIG. 4, below) to result in the best prediction of BL.

In another aspect, the effectiveness of hydration can be assessed by estimating or predicting the volume, V, of fluid necessary for effective hydration of the patient. This volume, V, can indicate a volume of fluid needed for full hydration if therapy has not yet begun, and/or it can indicate a volume remaining for fully effective hydration if therapy is underway. Like BL, the value of V can be estimated/predicted using the modeling techniques described herein and in the Related Applications. In a general case, V can be expressed as the following:

V=ƒ_{V}(CRIₜ,FVₜ,Sₜ) (Eq. 7)

where V is an estimated volume of fluid needed by a patient need to prevent over or under hydration, ƒ_{V}(CRIₜ, FVₜ, Sₜ) is an algorithm embodied by a model generated empirically, e.g., using the techniques described with respect to FIG. 4 below, and/or in the Related Applications, CRIₜ is a time history of CRI values, FVₜ is a time history of fluid volume being given to the patient, and Sₜ is a time history of physiological data received from the one or more sensors.

As with the estimate of BL, various embodiments can employ subsets of the parameters used in the general expression of Eq. 7. Thus, different embodiments might calculate V as follows:

V=ƒ_{V}(CRIₜ,FVₜ) (Eq. 8)

or

V=ƒ_{V}(FVₜ,Sₜ). (Eq. 9)

Yet another way of assessing effectiveness of hydration (which can even include assessing the need for hydration) is estimating the probability P_{ƒ} that the patient requires fluids; this probability can estimate the likelihood that the patient requires hydration if therapy has not been initiated, and/or, if hydration therapy is underway, the probability can estimate the likelihood that further hydration is necessary. The value of this probability, which can be expressed, e.g., as a percentage, as a decimal value between 0 and 1, etc. can be estimated using the following expression:

P_{ƒ}=ƒ_{P}_{_{ƒ}}(CRIₜ,Sₜ) (Eq. 10)

where P_{ƒ} is the estimated probability that the patient requires fluid, ƒ_{P}_{_{ƒ}}(CRIₜ, Sₜ) is a relationship derived based on empirical study, CRIₜ is a time history of CRI values, and Sₜ is a time history of physiological data received from the one or more sensors. Once again, this general expression can be employed, in various embodiments, using subsets of the parameters in the general expression, such as the following:

P_{ƒ}=ƒ_{P}_{_{ƒ}}(CRIₜ) (Eq. 11)

or

P_{ƒ}=ƒ_{P}_{_{ƒ}}(Sₜ) (Eq. 12)

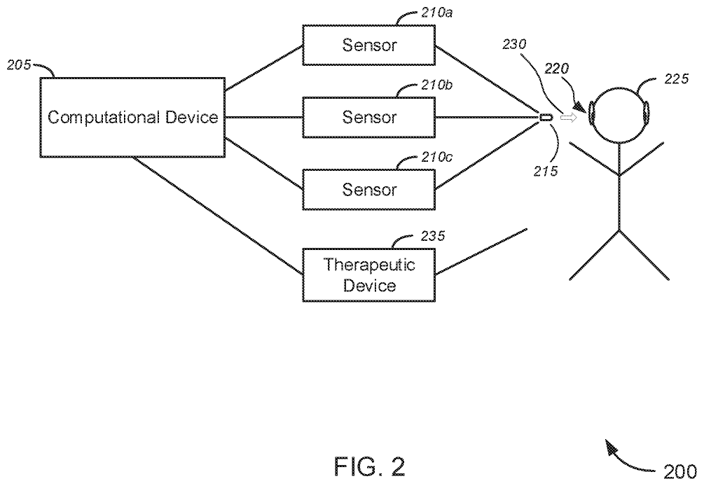

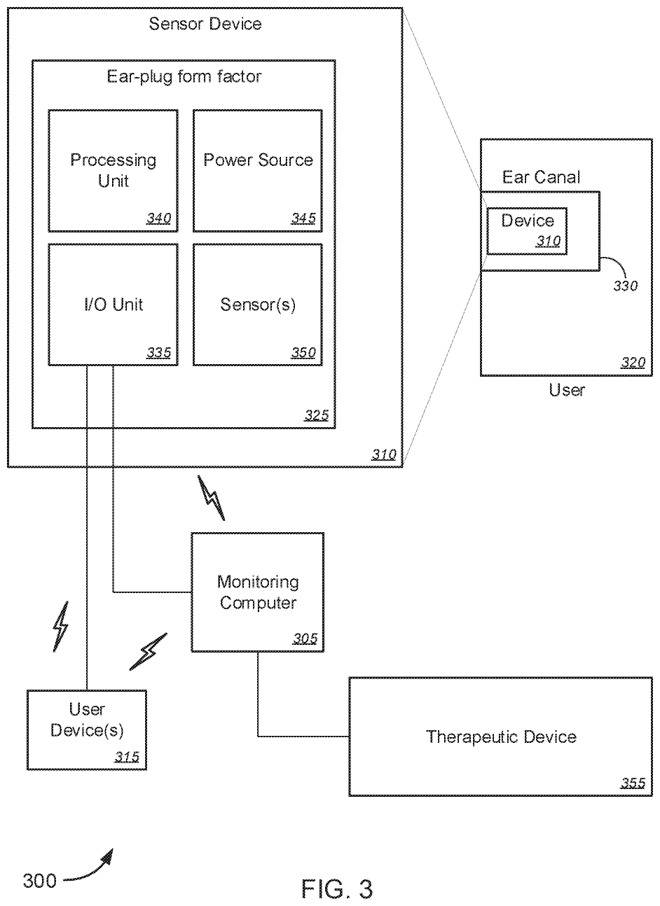

In the estimate of any of BL, V, or P_{ƒ}, the function ƒ expresses a relationship that is derived based on empirical study. In a set of embodiments, for example, various sensor data can be collected from test subjects before, during, and/or after hydration efforts, during hemorrhaging, or under other conditions that might simulate such situations. This sensor data can be analyzed to develop models, using techniques similar to those of FIG. 4 below, which can then be used to estimate various assessments of hydration effectiveness, using, e.g., the methods described below with respect to FIGS. 2 and 3.

A measure of CRI, BL, V, and/or P_{ƒ} can be useful in a variety of clinical settings, including, but not limited to: 1) acute blood loss volume due to injury or surgery; 2) acute circulatory volume loss due to hemodialysis (also called intradialytic hypotension); and 3) acute circulatory volume loss due to various causes of dehydration (e.g., reduced fluid intake, vomiting, dehydration, etc.). A change in CRI can also herald other conditions, including, without limitation, changes in blood pressure, general fatigue, overheating, and/or certain types of illnesses. Accordingly, the tools and techniques for estimating and/or predicting CRI can have a variety of applications in a clinical setting, including, without limitation, diagnosing such conditions.

Moreover, measures of CRI, BL, V, and/or P_{ƒ} can have applicability outside the clinical setting. For example, an athlete can be monitored (e.g., using a wrist-wearable hydration monitor) before, during, or after competition or training to ensure optimal performance (and overall health and recovery). In other situations, a person concerned about overall wellbeing can employ a similar hydration monitor to ensure that he or she is getting enough (but not too much) fluid, ill infants or adults can be monitored while ill to ensure that symptoms (e.g., vomiting, diarrhea, etc.) do not result in dehydration, and the like. Similarly, soldiers in the field (particularly in harsh conditions) can be monitored to ensure optimal operational readiness.

In various embodiments, a hydration monitor, compensatory reserve monitor, a wrist-wearable sensor device, and/or another integrated system can include, but is not limited to, some or all of the following functionality, as described in further detail herein and in the Related Applications:

A. Estimating and/or displaying intravascular volume loss to hemodynamic decompensation (or cardiovascular collapse).

B. Estimating, predicting, and/or displaying a patient's compensatory reserve as an index that is proportional to an approximate measure of intravascular volume loss to CV collapse, recognizing that each patient has a unique reserve capacity.

C. Estimating, predicting, and/or displaying a patient's compensatory reserve as an index with a normative value at euvolemia (for example, CRI=1), representing a state in which the patient is normovolemic; a minimum value (for example, CRI=0) which implies no circulatory reserve and that the patient is experiencing CV collapse; and/or an excess value (for example, CRI>1) representing a state in which the patient is hypervolemic; the patient's normalized compensatory reserve can be displayed on a continuum between the minimum and maximum values (perhaps labeled by different symbols and/or colors depending on where the patient falls on the continuum).

D. Determining and/or displaying a probability that bleeding or intravascular volume loss has occurred.

E. Displaying an indicator that intravascular volume loss has occurred and/or is ongoing; as well as other measures of reserve, such as trend lines.

F. Estimating a patient's current blood pressure and/or predicting a patient's future blood pressure.

G. Estimating the current effectiveness of fluid resuscitation efforts.

H. Predicting the future effectiveness of fluid resuscitation efforts.

I. Estimating and/or predicting a volume of fluid necessary for effective resuscitation.

J. Estimating a probability that a patient needs fluids.

K. Estimating a hydration state of a patient or user.

L. Predicting a future hydration state of a patient or user.

M. Estimating and/or predicting a volume of fluid intake necessary for adequate hydration of a patient or user.

N. Estimating a probability that a patient is dehydrated.

In various embodiments, CRI, BL, V, and/or P_{ƒ} estimates can be (i) based on a fixed time history of patient monitoring (for example a 30 second or 30 heart beat window); (ii) based on a dynamic time history of patient monitoring (for example monitoring for 200 minutes, the system may use all sensor information gathered during that time to refine and improve CRI estimates, hydration effectiveness assessments, etc.); (iii) based on either establishing baseline estimates when the patient is normovolemic (no volume loss has occurred); and/or (iv) based on NO baselines estimates when patient is normovolemic.

Certain embodiments can also recommend treatment options, based on the analysis of the patient's condition (including the estimated/predicted blood pressure, probability of bleeding, state of dehydration, and/or the patient's estimated and/or predicted CRI). Treatment options can include, without limitation, such things as optimizing hemodynamics, ventilator adjustments, IV fluid adjustments (e.g., controlling the flow rate of an IV pump or the drip rate of an IV drip), transfusion of blood or blood products, infusion of volume expanders, medication changes, changes in patient position, and/or surgical therapy, or the like.

As one example, certain embodiments can be used to control an IV drip, IV pump, or rapid infuser. For instance, an embodiment might estimate the probability that a patient requires fluids and might activate such a device in response to that estimate (or instruct a clinician to attach such a device to the patient and activate the device). The system might then monitor the progress of the hydration effort (through continual or periodic assessment of the effectiveness of hydration) and increase/decrease drip or flow rates accordingly.

As another example, certain embodiments can be used as an input for a hemodialysis procedure. For example, certain embodiments can predict how much intravascular (blood) volume can be safely removed from a patient during a hemodialysis process. For example, an embodiment might provide instructions to a human operator of a hemodialysis machine, based on estimates or predictions of the patient's CRI. Additionally and/or alternatively, such embodiments can be used to continuously self-adjust the ultra-filtration rate of the hemodialysis equipment, thereby completely avoiding intradialytic hypotension and its associated morbidity.

As yet another example, certain embodiments can be used to estimate and/or predict a dehydration state (and/or the amount of dehydration) in an individual (e.g., a trauma patient, an athlete, an elder living at home, etc.) and/or to provide treatment (either by providing recommendations to treating personnel or by directly controlling appropriate therapeutic equipment). For instance, if an analytical model indicates a relationship between CRI (and/or any other physiological phenomena that can be measured and/or estimated using the techniques described herein and in the Related Applications) and dehydration state, an embodiment can apply that model, using the techniques described herein, to estimate a dehydration state of the patient.

### Specific Exemplary Embodiments

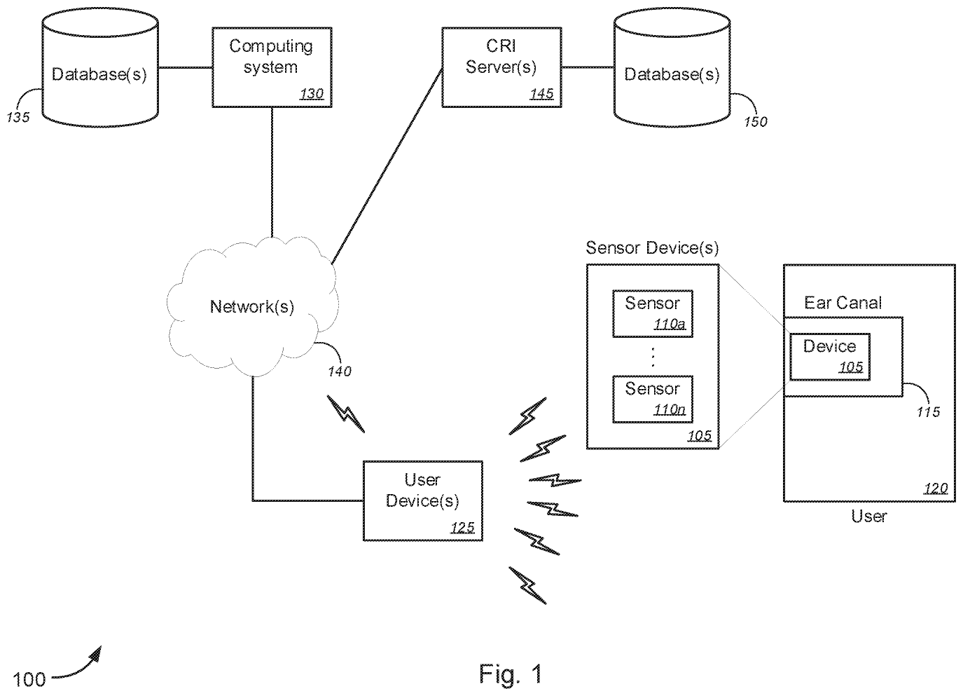

We now turn to the embodiments as illustrated by the drawings. FIGS. 1-5 illustrate some of the features of the method, system, and apparatus for implementing physiological monitoring, and, more particularly, to methods, systems, and apparatuses for implementing physiological state measurement of a user based on ear-based physiological data monitoring, as referred to above. The methods, systems, and apparatuses illustrated by FIGS. 1-5 refer to examples of different embodiments that include various components and steps, which can be considered alternatives or which can be used in conjunction with one another in the various embodiments. The description of the illustrated methods, systems, and apparatuses shown in FIGS. 1-5 is provided for purposes of illustration and should not be considered to limit the scope of the different embodiments.

With reference to the figures, FIG. 1 is a schematic diagram illustrating a system 100 for implementing physiological state measurement of a user based on ear-based physiological data monitoring, in accordance with various embodiments.

In the non-limiting embodiment of FIG. 1, system 100 might comprise one or more sensor devices 105, which might include, without limitation, one or more sensors 110a-110n (collectively, “sensors 110” or the like). According to some embodiments, the one or more sensors 110 might include, without limitation, at least one of one or more thermal sensors, one or more optical sensors, or one or more heart rate sensors, and/or the like. Alternatively, or additionally, the one or more sensors 110 might include, but are not limited to, one or more accelerometers, one or more gyroscopes, one or more location sensors, one or more compasses, or one or more altimeters, and/or the like. Alternatively, or additionally, the one or more sensors 110 might include, without limitation, at least one of one or more skin temperature sensors; one or more moisture sensors; one or more resistance sensors; one or more electrodermal activity (“EDA”) sensors; one or more body temperature sensors; one or more core temperature sensors; one or more fluid intake measurement sensors; one or more sensors measuring a compensatory reserve index (“CRI”) of the user; one or more sensors measuring hemodynamic status of the user; one or more sensors measuring closeness of hemodynamic collapse due to at least one of heat stress, hydration, or central fluid loss; one or more sensors that continuously capture one or more pulsatile components of a cardiac cycle of the user; one or more electrocardiograph sensors; or one or more respiration rate sensors; and/or the like. In some instances, the one or more sensors that continuously capture the one or more pulsatile components of the cardiac cycle of the user might include, without limitation, at least one of radio frequency (“RF”) sensor, a photoplethysmograph (“PPG”), a volume clamp, or a continuous blood pressure (“BP”) sensor, and/or the like. In some cases, the one or more sensors 110 might be encapsulated within the one or more sensor devices 105, which might comprise an ear-plug-shaped housing configured to fit within an ear canal 115 of a user 120.

In some embodiments, system 100 might further comprise one or more user devices 125. In some cases, the user device(s) 125 might each include, without limitation, one of a smart phone, a mobile phone, a smart watch, a tablet computer, a laptop computer, a desktop computer, or a dedicated sensor control device, and/or the like.

According to some embodiments, system 100 might further comprise a (remote) computing system 130 and corresponding database(s) 135 that communicatively couples to the user device(s) 125 and/or at least one of sensor device(s) 105 and/or sensor(s) 110, via network(s) 140. In some cases, system 100 might further comprise CRI server(s) 145 and corresponding database(s) 150 that may communicatively couple to at least one of the computing system 130, the user device(s) 125, the sensor device(s) 105, and/or the sensor(s) 110, via network(s) 140. According to various embodiments, the user device(s) 125 might communicatively couple to each of at least one of network(s) 140, sensor device(s) 105, and/or sensor(s) 110, via wireless communications systems or technologies (including, but not limited to, Bluetooth™ communications, Z-wave communications, ZigBee communications, XBee communications, or WiFi communications, and/or the like), as denoted in FIG. 1 by the lightning bolt symbols. In some cases, the network(s) 140 might include a local area network (“LAN”), including, without limitation, a fiber network, an Ethernet network, a Token-Ring™ network, and/or the like; a wide-area network (“WAN”); a wireless wide area network (“WWAN”); a virtual network, such as a virtual private network (“VPN”); the Internet; an intranet; an extranet; a public switched telephone network (“PSTN”); an infra-red network; a wireless network, including, without limitation, a network operating under any of the IEEE 802.11 suite of protocols, the Bluetooth™ protocol known in the art, the Z-Wave protocol known in the art, the ZigBee protocol or other IEEE 802.15.4 suite of protocols known in the art, and/or any other wireless protocol; and/or any combination of these and/or other networks. In a particular embodiment, the network might include an access network of the service provider (e.g., an Internet service provider (“ISP”)). In another embodiment, the network might include a core network of the service provider, and/or the Internet.

In operation, the one or more sensors 110—which is disposed in sensor device(s) 105 that is positioned at least partially within or inserted at least partially within the ear canal 115 of user 120—might monitor physiological data of the user 120, and might send data regarding the monitored physiological data of the user 120 as obtained from within the ear canal 115 of the user 120 to user device(s) 125 and/or computing system 130 (collectively, “computing system” or the like). The computing system might receive the data regarding the monitored physiological data of the user 120 as obtained from within the ear canal 115 of the user 120; might analyze the received data regarding the monitored physiological data of the user 120 as obtained from within the ear canal 115 of the user 120; might determine at least one physiological state of the user 120, based at least in part on the analysis of the data regarding the monitored physiological data of the user 120 as obtained from within the ear canal 115 of the user 120; and might send the determined at least one physiological state of the user 120 to the user device(s) 125 of the user 120 for display on a display screen of the user device(s) 125.

In some embodiments, monitoring the physiological data of the user might comprise monitoring a thermal reading from within the ear canal 115 of the user 120. In such cases, determining the at least one physiological state of the user might comprise determining, with the computing system, a body temperature of the user 120, based at least in part on the monitored thermal reading.

Alternatively, or additionally, monitoring the physiological data of the user might comprise monitoring a pulsatile waveform that is detected in the ear canal 115 of the user 120. In such cases, determining the at least one physiological state of the user might comprise calculating, with the computing system, at least one of a compensatory reserve index (“CRI”) or a heart rate (“HR”) of the user 120 based at least in part on the monitored pulsatile waveform.

In some embodiments, the one or more sensors 110a-110n may further include a microphone. The microphone may be configured to verbally query a physiological state of the user. Thus, in some embodiments, in response to receiving verbal command, the microphone may receive the verbal command, and transmit the verbal command to one or more of the user devices 125 and/or computing system 130 for further processing. For example, in response to receiving the response, the one or more sensor devices 105 may further be configured to begin detecting and/or monitoring physiological data as described above.

In some further embodiments, one or more sensor devices 105 may be coupled to each respective ear canal 115 of the user 120. For example, a first set of the one or more sensor devices 105 may be coupled to a first ear canal 115 (e.g., left ear canal) of the user 120, and a second set of the one or more sensor devices 105 may be coupled to a second ear canal 115 (e.g., right ear canal) of the user 120. By having a respective set of sensors located in each ear canal 115, higher fidelity signals may be obtained by allowing signals to be measured at two different points of reference. For example, in some embodiments, each of the first set and second set of one or more sensor devices 105 may include a respective subset of sensors 110a-110n, which may allow concurrent determination of respective monitored thermal reading and/or monitored pulsatile waveforms. Moreover, having a respective set of one or more sensor devices 105 in each respective ear canal 115 may further allow for detection of physiologic conditions such as fainting, and to determine the amount and direction of head tilting. In some examples, the respective sets of one or more sensors devices 105 may further be configured to sound an auditory alert if one of the sets of sensor devices 105 and/or a respective sensor 110a-110n of the respective set of sensor devices 105 is lost, mispositioned, or malfunctions.

In some further embodiments, respective sets of one or more sensor devices 105 may be configured to generate motion data indicative of the movement of the user 120. Accordingly, in some embodiments, the motion artifacts may be mitigated from each of the respectively monitored pulsatile waveforms. Motion artifacts may include, for example, noise and/or error introduced in the pulsatile waveform by the user's movement. For example, in some embodiments, motion data from the first, second, or a combination of first and second sets of one or more sensor devices 105 may be used to mitigate motion artifacts in pulsatile waveform data monitored by the first and/or second set of one or more sensor devices 105.

Merely by way of example, according to some embodiments, displaying the determined at least one physiological state of the user might comprise at least one of: displaying, on the display screen of the user device, an indication that the sensor device has been properly placed within the ear canal to sufficiently capture sensor readings; displaying, on the display screen of the user device, an indication that the sensor device has not been properly placed within the ear canal to sufficiently capture sensor readings and a set of instructions to properly insert the sensor device within the ear canal after removal of the sensor device from the ear canal; displaying, on the display screen of the user device, a body temperature of the user as measured by the one or more first sensors from within the ear canal of the user; displaying, on the display screen of the user device, a compensatory reserve index (“CRI”) as calculated based at least in part on pulsatile waveform readings as obtained by the one or more first sensors from within the ear canal of the user; displaying, on the display screen of the user device, a heart rate (“HR”) of the user as measured by the one or more first sensors from within the ear canal of the user; displaying, on the display screen of the user device, at least one notification indicating that one or more of CRI, HR, or body temperature readings are valid and complete; or displaying, on the display screen of the user device, one or more error messages when at least one of an error occurs or no valid readings can be made; and/or the like.

Correspondingly, in some embodiments, the determined at least one physiological state of the user might be audibly presented to the user (e.g., using one or more speakers of user device(s) 125, or the like). In some cases, audibly presenting the determined at least one physiological state of the user might comprise at least one of: audibly presenting, using one or more speakers of the user device, an indication that the sensor device has been properly placed within the ear canal to sufficiently capture sensor readings; audibly presenting, using the one or more speakers of the user device, an indication that the sensor device has not been properly placed within the ear canal to sufficiently capture sensor readings and a set of instructions to properly insert the sensor device within the ear canal after removal of the sensor device from the ear canal; audibly presenting, using the one or more speakers of the user device, a body temperature of the user as measured by the one or more first sensors from within the ear canal of the user; audibly presenting, using the one or more speakers of the user device, a compensatory reserve index (“CRI”) as calculated based at least in part on pulsatile waveform readings as obtained by the one or more first sensors from within the ear canal of the user; audibly presenting, using the one or more speakers of the user device, a heart rate (“HR”) of the user as measured by the one or more first sensors from within the ear canal of the user; audibly presenting, using the one or more speakers of the user device, one or more beeps or tones indicating that one or more of CRI, HR, or body temperature readings are valid and complete; or audibly presenting, using the one or more speakers of the user device, one or more error messages when at least one of an error occurs or no valid readings can be made; and/or the like.

According to some aspects, a measurement of compensatory reserve, as characterized by a compensatory reserve index (which is described in detail in the Related Applications, which have already been incorporated herein by reference in their entirety for all purposes), may be indicative of a body state of the user 120, as measured from within the ear canal of the user. In particular, the inventors of this application have found that physical activity or a change in physical activity of the user 120 (e.g., a body state (as described above) or a transition between body states (as also described above)) may result in an impulse response in the CRI of the user. Profiles of the CRI (as compared with base measurements of CRI of the individual user or a compilation of measurements of CRI across a sample of multiple users) may be indicative of health, fitness, and/or other physiological states of the user. In such cases, a CRI server (e.g., CRI server(s) 145 or the like) or other computational device might monitor physiological data of the user 120 (e.g., by using sensors, including, but not limited to the one or more first sensors, as described above) to measure a CRI of the user 120, and might further analyze the measured CRI to determine a body state of the user 120 and/or a transition between body states of the user 120, and might also further analyze the measured CRI to determine the physiological state of the user—including, but not limited to, at least one of a hydration state of the user, a dehydration state of the user, a fitness state of the user, a health state of the user, an exertion readiness state of the user, a fatigue state of the user, an alertness level of the user, an altitude sickness state of the user, a level of tolerance to heat of the user, a level of tolerance to cold of the user, a level of tolerance to other environmental conditions of the user, a level of tolerance to liquid limitations of the user, a level of tolerance to blood loss of the user, or one or more states of illness of the user (including, but not limited to, flu, cold, viral infection, bacterial infection, sepsis, heart disease, and/or the like), and/or the like. Such physiological states may then be presented to the user 120 (or a physician or other healthcare provider of the user 120) using a user interface of a user device (e.g., user device(s) 125, or the like), a display screen of a user device (e.g., user device(s) 125, or the like), a web portal, a software application (“app”), and/or the like.

In some embodiments, the physiological state determination may be performed in real-time or near-real-time based on the monitored sensor data (i.e., physiological data collected or monitored by the one or more first sensors).

These and other functions of the system 100 (and its components) are described in greater detail below with respect to FIGS. 2-4.

FIG. 2 is a schematic diagram illustrating a system 200 for estimating compensatory reserve, which can be used for implementing physiological state measurement of a user based on ear-based physiological data monitoring, in accordance with various embodiments.

FIG. 2 provides a general overview of a system 200 provided by certain embodiments. The system might include a computer system or computational device 205 in communication with one or more sensors 210 (which might include sensors 210a, 210b, and 210c, or the like), each of which might be disposed in a sensor device 215 that may be at least partially displaced or inserted within an ear canal 220 of a user 225 (as denoted by arrow 230). Each of the one or more sensors 210 might be configured to obtain physiological data from the subject (e.g., animal or human test subject or patient) 225. In one embodiment, the computer system 205 might comprise a Lenovo THINKPAD X200, 4 GB of RAM with Microsoft WINDOWS 7 operating system and might be programmed with software to execute the computational methods outlined herein or in the Related Applications. The computational methods can be implemented in MATLAB 2009b and C++ programming languages. A more general example of a computer system 205 that can be used in some embodiments is described in further detail below. Even more generally, however, the computer system 205 can be any system of one or more computers that are capable of performing the techniques described herein. In a particular embodiment, for example, the computer system 205 is capable of reading values from the physiological sensors 210; generating models of physiological state from those sensors; employing such models to make individual-specific estimations, predictions, or other diagnoses; displaying the results; recommending and/or implementing a therapeutic treatment as a result of the analysis; and/or archiving (learning) these results for use in future, model building and predictions; or the like.

The sensors 210 can be any of a variety of sensors (including, without limitation, those described herein) for obtaining physiological data from the subject. An exemplary sensor suite might include a Finometer sensor for obtaining a noninvasive continuous blood pressure waveform, a pulse oximeter sensor, an Analog to Digital Board (National Instruments USB-9215A 16-Bit, 4 channel) for connecting the sensors (either the pulse oximeter and/or the finometer) to the computer system 205. More generally, in an embodiment, one or more sensors 210 might obtain, e.g., using one or more of the techniques described herein, continuous physiological waveform data, such as continuous blood pressure. Input from the sensors 210 can constitute continuous data signals and/or outcomes that can be used to generate, and/or can be applied to, a predictive model as described below.

Merely by way of example, the one or more sensors 210 might include, but are not limited to, at least one of one or more thermal sensors, one or more optical sensors, or one or more heart rate sensors, and/or the like. Alternatively, or additionally, the one or more sensors 210 might include, without limitation, at least one of one or more accelerometers, one or more gyroscopes, one or more location sensors, one or more compasses, or one or more altimeters, and/or the like. Alternatively, or additionally, the one or more sensors 210 might include, but are not limited to, at least one of one or more skin temperature sensors; one or more moisture sensors; one or more resistance sensors; one or more electrodermal activity (“EDA”) sensors; one or more body temperature sensors; one or more core temperature sensors; one or more fluid intake measurement sensors; one or more sensors measuring a compensatory reserve index (“CRI”) of the user; one or more sensors measuring hemodynamic status of the user; one or more sensors measuring closeness of hemodynamic collapse due to at least one of heat stress, hydration, or central fluid loss; one or more sensors that continuously capture one or more pulsatile components of a cardiac cycle of the user; one or more electrocardiograph sensors; or one or more respiration rate sensors; and/or the like. In some instances, the one or more sensors that continuously capture the one or more pulsatile components of the cardiac cycle of the user might include, without limitation, at least one of radio frequency (“RF”) sensor, a photoplethysmograph (“PPG”), a volume clamp, or a continuous blood pressure (“BP”) sensor, and/or the like. In some cases, the one or more sensors 210 might be encapsulated within the one or more sensor devices 215, which might comprise an ear-plug-shaped housing configured to fit within an ear canal 220 of a user 225.

In some cases, the structure or system might include a therapeutic device 235 (also referred to herein as a “physiological assistive device”), which can be controlled by the computer system 205 to administer therapeutic treatment, in accordance with the recommendations developed by analysis of a patient's physiological data. In a particular embodiment, the therapeutic device might comprise hemodialysis equipment (also referred to as a hemodialysis machine), which can be controlled by the computer system 205 based on the estimated CRI of the patient, as described in further detail below. Further examples of therapeutic devices in other embodiments can include a cardiac assist device, a ventilator, an automatic implantable cardioverter defibrillator (“AICD”), pacemakers, an extracorporeal membrane oxygenation circuit, a positive airway pressure (“PAP”) device (including, without limitation, a continuous positive airway pressure (“cPAP”) device, or the like), an anesthesia machine, an integrated critical care system, a medical robot, intravenous and/or intra-arterial pumps that can provide fluids and/or therapeutic compounds (e.g., through intravenous injection), intravenous drips, a rapid infuser, a heating/cooling blanket, and/or the like.

System 200 of FIG. 2 might otherwise be implemented in a similar manner as described in detail herein with respect to system 100 of FIG. 1 or method 400 of FIG. 4.

FIG. 3 is a schematic diagram illustrating an example 300 of a sensor system that can be worn on a patient's body and that can be used for implementing physiological state measurement of a user based on ear-based physiological data monitoring, in accordance with various embodiments.

FIG. 3 illustrates in more detail an exemplary sensor device 310, which can be used in the system 300. (It should be noted, of course, that the depicted sensor device 310 of FIG. 3 is not intended to be limiting, and different embodiments can employ any sensor that captures suitable data, including, without limitation, sensors described elsewhere in this disclosure and in the Related Applications.) The illustrated sensor device 310 is designed to have an ear-plug form factor 325 that is designed to be at least partially displaced or inserted within the ear canal 330 of user 320. The illustrated sensor device 310 can be used both in clinical settings and in the field (e.g., on any person for whom monitoring might be beneficial, for a variety of reasons, including, without limitation, assessment of blood pressure and/or hydration during athletic competition or training, daily activities, military training or action, etc.). In one aspect, the sensor device 310 can serve as an integrated hydration monitor, which can assess hydration as described in some of the Related Applications, display an indication of the assessment, recommend therapeutic action based on the assessment, or the like, in a form factor that can be worn during athletic events and/or daily activities. Alternatively, or additionally, the sensor device 310 can serve to monitor maneuvers (or motions) of the patient as well as activities of the patient to identify or determine a body state or transition between body states of the patient, and subsequently to determine a physiological state of the patient based at least in part on the determined body state or transition between body states of the patient (and perhaps also based at least in part on data, e.g., CRI data, of the patient and/or of a plurality of other patients or users), as described in detail in the 0463.18PR Application (which has been incorporated herein by reference in its entirety for all purposes). In other words, the system might be able to determine physiological states of the patient, including, but not limited to, at least one of a hydration state of the user, a dehydration state of the user, a fitness state of the user, a health state of the user, an exertion readiness state of the user, a fatigue state of the user, an alertness level of the user, an altitude sickness state of the user, a level of tolerance to heat of the user, a level of tolerance to cold of the user, a level of tolerance to other environmental conditions of the user, a level of tolerance to liquid limitations of the user, a level of tolerance to blood loss of the user, or one or more states of illness of the user (including, but not limited to, flu, cold, viral infection, bacterial infection, sepsis, heart disease, and/or the like), and/or the like. Alternatively, or additionally, the sensor device 310 might detect thermal readings and pulsatile waveform readings from within the ear canal 330 of user 320. The detected thermal readings may be used to calculate a body temperature of the user 320, while the detected pulsatile waveform readings may be used to calculate CRI and heart rate of the user 320.

Hence, the exemplary sensor 310 device (e.g., physiological monitor or the like) might include an ear-plug form factor or housing 325. The sensor with the ear-plug form factor or housing 325 might comprise an input/output (“I/O”) unit 335, a processing unit 340, a power source 345 (e.g., a battery or the like), and one or more sensors 350. Merely by way of example, the one or more sensors 350 might include, but are not limited to, at least one of one or more thermal sensors, one or more optical sensors, or one or more heart rate sensors, and/or the like. Alternatively, or additionally, the one or more sensors 350 might include, without limitation, at least one of one or more accelerometers, one or more gyroscopes, one or more location sensors, one or more compasses, or one or more altimeters, and/or the like. Alternatively, or additionally, the one or more sensors 350 might include, but are not limited to, at least one of one or more skin temperature sensors; one or more moisture sensors; one or more resistance sensors; one or more electrodermal activity (“EDA”) sensors; one or more body temperature sensors; one or more core temperature sensors; one or more fluid intake measurement sensors; one or more sensors measuring a compensatory reserve index (“CRI”) of the user; one or more sensors measuring hemodynamic status of the user; one or more sensors measuring closeness of hemodynamic collapse due to at least one of heat stress, hydration, or central fluid loss; one or more sensors that continuously capture one or more pulsatile components of a cardiac cycle of the user; one or more electrocardiograph sensors; or one or more respiration rate sensors; and/or the like. In some instances, the one or more sensors that continuously capture the one or more pulsatile components of the cardiac cycle of the user might include, without limitation, at least one of radio frequency (“RF”) sensor, a photoplethysmograph (“PPG”), a volume clamp, or a continuous blood pressure (“BP”) sensor, and/or the like.

The sensor device 310 might communicate, via I/O unit 335, with monitoring computer 305 and/or with user device(s) 315. Such communication can be wired (e.g., via a standard—such as USB—or proprietary connector on the housing 325) and/or wireless (e.g., via Bluetooth, such as Bluetooth Low Energy (“BTLE”), near field connection (“NFC”), WiFi, ZigBee, XBee, Z-Wave, or any other suitable radio technology).

In different embodiments, the processing unit 340 can have different types of functionality. For example, in some cases, the processing unit 340 might simply act to store and/or organize data prior to transmitting the data through the I/O unit 335 to the monitoring computer 305, which might perform data analysis, to control a therapeutic device 355 (as described in some of the Related Applications, or the like), etc. In other cases, however, the processing unit 340 might act as a specialized computer (e.g., with some or all of the components described in connection with FIG. 1 above, and/or some or all of the functionality ascribed to the computer 205 of FIG. 2, or the like), such that the processing unit 340 can perform data analysis onboard, e.g., to estimate and/or predict a patient's current and/or future physiological state. In some cases, the user device(s) 315 might include a display (not shown), which can display any output described herein, including, without limitation, estimated and/or predicted values (e.g., of CRI, blood pressure, hydration status, or physiological state in general, etc.), data captured by the sensor (e.g., heart rate, body temperature, pulse oximetry data, etc.), and/or the like.

Once again, the embodiment illustrated by FIG. 3 should be considered only illustrative, and not limiting, in nature. System 300 of FIG. 3 might otherwise be implemented in a similar manner as described in detail herein with respect to system 100 of FIG. 1 or method 400 of FIG. 4.

FIGS. 4A-4C (collectively, “FIG. 4”) are flow diagrams illustrating a method for implementing physiological state measurement of a user based on ear-based physiological data monitoring, in accordance with various embodiments.

While the techniques and procedures are depicted and/or described in a certain order for purposes of illustration, it should be appreciated that certain procedures may be reordered and/or omitted within the scope of various embodiments. Moreover, while the method 400 illustrated by FIG. 4 can be implemented by or with (and, in some cases, are described below with respect to) the systems, examples, or embodiments 100, 200, and 300 of FIGS. 1, 2, and 3, respectively (or components thereof), such methods may also be implemented using any suitable hardware (or software) implementation. Similarly, while each of the systems, examples, or embodiments 100, 200, and 300 of FIGS. 1, 2, and 3, respectively (or components thereof), can operate according to the method 400 illustrated by FIG. 4 (e.g., by executing instructions embodied on a computer readable medium), the systems, examples, or embodiments 100, 200, and 300 of FIGS. 1, 2, and 3 can each also operate according to other modes of operation and/or perform other suitable procedures.

In the non-limiting embodiment of FIG. 4A, method 400, at block 405, might comprise monitoring, with one or more first sensors disposed in a sensor device that is at least partially displaced within an ear canal of a user, physiological data of the user. At block 410, method 400 might comprise sending, with the one or more first sensors and to a computing system, data regarding the monitored physiological data of the user as obtained from within the ear canal of the user. Method 400 might further comprise, at block 415, receiving, with the computing system, the data regarding the monitored physiological data of the user as obtained from within the ear canal of the user. Method 400 might further comprise analyzing, with the computing system, the received data regarding the monitored physiological data of the user as obtained from within the ear canal of the user (block 420) and determining, with the computing system, at least one physiological state of the user, based at least in part on the analysis of the data regarding the monitored physiological data of the user as obtained from within the ear canal of the user (block 425).

Method 400 might further comprise sending, with the computing system and to a user device, the determined at least one physiological state of the user (block 430) and displaying, on a display screen of the user device, the determined at least one physiological state of the user (block 435). In some embodiments, method 400, at optional block 440, might comprise audibly presenting, using one or more speakers of the user device, the determined at least one physiological state of the user.

In some embodiments, the one or more first sensors might each include, but is not limited to, at least one of one or more thermal sensors, one or more optical sensors, or one or more heart rate sensors, and/or the like. In some cases, the one or more first sensors might be encapsulated within a sensor device, where the sensor device might comprise an ear-plug-shaped housing configured to fit within the ear canal of the user. In some instances, the user device might include, without limitation, one of a smart phone, a mobile phone, a smart watch, a tablet computer, a laptop computer, a desktop computer, or a dedicated sensor control device, and/or the like.

Turning to FIG. 4B, monitoring the physiological data of the user (at block 405) might comprise monitoring a thermal reading from within the ear canal of the user (block 445). In such cases, determining the at least one physiological state of the user (at block 425) might comprise determining, with the computing system, a body temperature of the user, based at least in part on the monitored thermal reading from within the ear canal of the user (block 450).

Alternatively, or additionally, with reference to FIG. 4C, monitoring the physiological data of the user (at block 405) might comprise monitoring a pulsatile waveform that is detected in the ear canal of the user (block 455). In such cases, determining the at least one physiological state of the user (at block 425) might comprise calculating, with the computing system, at least one of a compensatory reserve index (“CRI”) or a heart rate (“HR”) of the user based at least in part on the monitored pulsatile waveform (block 460).

Merely by way of example, according to some embodiments, displaying the determined at least one physiological state of the user (at block 435) might comprise at least one of: displaying, on the display screen of the user device, an indication that the sensor device has been properly placed within the ear canal to sufficiently capture sensor readings; displaying, on the display screen of the user device, an indication that the sensor device has not been properly placed within the ear canal to sufficiently capture sensor readings and a set of instructions to properly insert the sensor device within the ear canal after removal of the sensor device from the ear canal; displaying, on the display screen of the user device, a body temperature of the user as measured by the one or more first sensors from within the ear canal of the user; displaying, on the display screen of the user device, a compensatory reserve index (“CRI”) as calculated based at least in part on pulsatile waveform readings as obtained by the one or more first sensors from within the ear canal of the user; displaying, on the display screen of the user device, a heart rate (“HR”) of the user as measured by the one or more first sensors from within the ear canal of the user; displaying, on the display screen of the user device, at least one notification indicating that one or more of CRI, HR, or body temperature readings are valid and complete; or displaying, on the display screen of the user device, one or more error messages when at least one of an error occurs or no valid readings can be made; and/or the like.

Correspondingly, in some embodiments, audibly presenting the determined at least one physiological state of the user (at optional block 435) might comprise at least one of: audibly presenting, using one or more speakers of the user device, an indication that the sensor device has been properly placed within the ear canal to sufficiently capture sensor readings; audibly presenting, using the one or more speakers of the user device, an indication that the sensor device has not been properly placed within the ear canal to sufficiently capture sensor readings and a set of instructions to properly insert the sensor device within the ear canal after removal of the sensor device from the ear canal; audibly presenting, using the one or more speakers of the user device, a body temperature of the user as measured by the one or more first sensors from within the ear canal of the user; audibly presenting, using the one or more speakers of the user device, a compensatory reserve index (“CRI”) as calculated based at least in part on pulsatile waveform readings as obtained by the one or more first sensors from within the ear canal of the user; audibly presenting, using the one or more speakers of the user device, a heart rate (“HR”) of the user as measured by the one or more first sensors from within the ear canal of the user; audibly presenting, using the one or more speakers of the user device, one or more beeps or tones indicating that one or more of CRI, HR, or body temperature readings are valid and complete; or audibly presenting, using the one or more speakers of the user device, one or more error messages when at least one of an error occurs or no valid readings can be made; and/or the like.

Exemplary System and Hardware Implementation

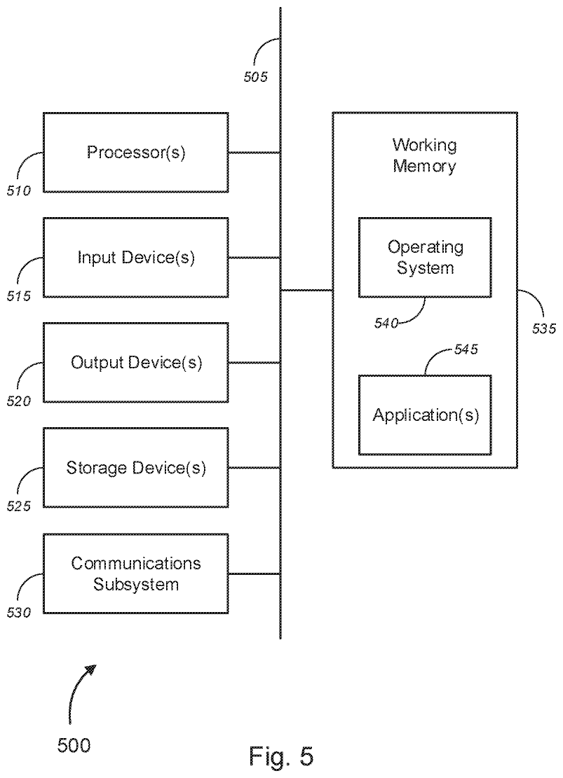

FIG. 5 is a block diagram illustrating an exemplary computer or system hardware architecture, in accordance with various embodiments. FIG. 5 provides a schematic illustration of one embodiment of a computer system 500 of the service provider system hardware that can perform the methods provided by various other embodiments, as described herein, and/or can perform the functions of computer or hardware system (i.e., sensor devices 105 and 310, user devices 120, computing system 130, computational device 205, monitoring computer 305, compensatory reserve index (“CRI”) server(s) 145, and therapeutic devices 215 and 315, etc.), as described above. It should be noted that FIG. 5 is meant only to provide a generalized illustration of various components, of which one or more (or none) of each may be utilized as appropriate. FIG. 5, therefore, broadly illustrates how individual system elements may be implemented in a relatively separated or relatively more integrated manner.

The computer or hardware system 500—which might represent an embodiment of the computer or hardware system (i.e., sensor devices 105 and 310, user devices 120, computing system 130, computational device 205, monitoring computer 305, CRI server(s) 145, and therapeutic devices 215 and 315, etc.), described above with respect to FIGS. 1-4—is shown comprising hardware elements that can be electrically coupled via a bus 505 (or may otherwise be in communication, as appropriate). The hardware elements may include one or more processors 510, including, without limitation, one or more general-purpose processors and/or one or more special-purpose processors (such as microprocessors, digital signal processing chips, graphics acceleration processors, and/or the like); one or more input devices 515, which can include, without limitation, a mouse, a keyboard, and/or the like; and one or more output devices 520, which can include, without limitation, a display device, a printer, and/or the like.

The computer or hardware system 500 may further include (and/or be in communication with) one or more storage devices 525, which can comprise, without limitation, local and/or network accessible storage, and/or can include, without limitation, a disk drive, a drive array, an optical storage device, solid-state storage device such as a random access memory (“RAM”) and/or a read-only memory (“ROM”), which can be programmable, flash-updateable, and/or the like. Such storage devices may be configured to implement any appropriate data stores, including, without limitation, various file systems, database structures, and/or the like.

The computer or hardware system 500 might also include a communications subsystem 530, which can include, without limitation, a modem, a network card (wireless or wired), an infra-red communication device, a wireless communication device and/or chipset (such as a Bluetooth™ device, an 802.11 device, a WiFi device, a WiMax device, a WWAN device, cellular communication facilities, etc.), and/or the like. The communications subsystem 530 may permit data to be exchanged with a network (such as the network described below, to name one example), with other computer or hardware systems, and/or with any other devices described herein. In many embodiments, the computer or hardware system 500 will further comprise a working memory 535, which can include a RAM or ROM device, as described above.

The computer or hardware system 500 also may comprise software elements, shown as being currently located within the working memory 535, including an operating system 540, device drivers, executable libraries, and/or other code, such as one or more application programs 545, which may comprise computer programs provided by various embodiments (including, without limitation, hypervisors, VMs, and the like), and/or may be designed to implement methods, and/or configure systems, provided by other embodiments, as described herein. Merely by way of example, one or more procedures described with respect to the method(s) discussed above might be implemented as code and/or instructions executable by a computer (and/or a processor within a computer); in an aspect, then, such code and/or instructions can be used to configure and/or adapt a general purpose computer (or other device) to perform one or more operations in accordance with the described methods.

A set of these instructions and/or code might be encoded and/or stored on a non-transitory computer readable storage medium, such as the storage device(s) 525 described above. In some cases, the storage medium might be incorporated within a computer system, such as the system 500. In other embodiments, the storage medium might be separate from a computer system (i.e., a removable medium, such as a compact disc, etc.), and/or provided in an installation package, such that the storage medium can be used to program, configure, and/or adapt a general purpose computer with the instructions/code stored thereon. These instructions might take the form of executable code, which is executable by the computer or hardware system 500 and/or might take the form of source and/or installable code, which, upon compilation and/or installation on the computer or hardware system 500 (e.g., using any of a variety of generally available compilers, installation programs, compression/decompression utilities, etc.) then takes the form of executable code.

It will be apparent to those skilled in the art that substantial variations may be made in accordance with specific requirements. For example, customized hardware (such as programmable logic controllers, field-programmable gate arrays, application-specific integrated circuits, and/or the like) might also be used, and/or particular elements might be implemented in hardware, software (including portable software, such as applets, etc.), or both. Further, connection to other computing devices such as network input/output devices may be employed.

As mentioned above, in one aspect, some embodiments may employ a computer or hardware system (such as the computer or hardware system 500) to perform methods in accordance with various embodiments of the invention. According to a set of embodiments, some or all of the procedures of such methods are performed by the computer or hardware system 500 in response to processor 510 executing one or more sequences of one or more instructions (which might be incorporated into the operating system 540 and/or other code, such as an application program 545) contained in the working memory 535. Such instructions may be read into the working memory 535 from another computer readable medium, such as one or more of the storage device(s) 525. Merely by way of example, execution of the sequences of instructions contained in the working memory 535 might cause the processor(s) 510 to perform one or more procedures of the methods described herein.

The terms “machine readable medium” and “computer readable medium,” as used herein, refer to any medium that participates in providing data that causes a machine to operate in a specific fashion. In an embodiment implemented using the computer or hardware system 500, various computer readable media might be involved in providing instructions/code to processor(s) 510 for execution and/or might be used to store and/or carry such instructions/code (e.g., as signals). In many implementations, a computer readable medium is a non-transitory, physical, and/or tangible storage medium. In some embodiments, a computer readable medium may take many forms, including, but not limited to, non-volatile media, volatile media, or the like. Non-volatile media includes, for example, optical and/or magnetic disks, such as the storage device(s) 525. Volatile media includes, without limitation, dynamic memory, such as the working memory 535. In some alternative embodiments, a computer readable medium may take the form of transmission media, which includes, without limitation, coaxial cables, copper wire, and fiber optics, including the wires that comprise the bus 505, as well as the various components of the communication subsystem 530 (and/or the media by which the communications subsystem 530 provides communication with other devices). In an alternative set of embodiments, transmission media can also take the form of waves (including without limitation radio, acoustic, and/or light waves, such as those generated during radio-wave and infra-red data communications).

Common forms of physical and/or tangible computer readable media include, for example, a floppy disk, a flexible disk, a hard disk, magnetic tape, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave as described hereinafter, or any other medium from which a computer can read instructions and/or code.

Various forms of computer readable media may be involved in carrying one or more sequences of one or more instructions to the processor(s) 510 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer or hardware system 500. These signals, which might be in the form of electromagnetic signals, acoustic signals, optical signals, and/or the like, are all examples of carrier waves on which instructions can be encoded, in accordance with various embodiments of the invention.

The communications subsystem 530 (and/or components thereof) generally will receive the signals, and the bus 505 then might carry the signals (and/or the data, instructions, etc. carried by the signals) to the working memory 535, from which the processor(s) 505 retrieves and executes the instructions. The instructions received by the working memory 535 may optionally be stored on a storage device 525 either before or after execution by the processor(s) 510.

While certain features and aspects have been described with respect to exemplary embodiments, one skilled in the art will recognize that numerous modifications are possible. For example, the methods and processes described herein may be implemented using hardware components, software components, and/or any combination thereof. Further, while various methods and processes described herein may be described with respect to particular structural and/or functional components for ease of description, methods provided by various embodiments are not limited to any particular structural and/or functional architecture but instead can be implemented on any suitable hardware, firmware and/or software configuration. Similarly, while certain functionality is ascribed to certain system components, unless the context dictates otherwise, this functionality can be distributed among various other system components in accordance with the several embodiments.

Moreover, while the procedures of the methods and processes described herein are described in a particular order for ease of description, unless the context dictates otherwise, various procedures may be reordered, added, and/or omitted in accordance with various embodiments. Moreover, the procedures described with respect to one method or process may be incorporated within other described methods or processes; likewise, system components described according to a particular structural architecture and/or with respect to one system may be organized in alternative structural architectures and/or incorporated within other described systems. Hence, while various embodiments are described with—or without—certain features for ease of description and to illustrate exemplary aspects of those embodiments, the various components and/or features described herein with respect to a particular embodiment can be substituted, added and/or subtracted from among other described embodiments, unless the context dictates otherwise. Consequently, although several exemplary embodiments are described above, it will be appreciated that the invention is intended to cover all modifications and equivalents within the scope of the following claims.

What is claimed is:

1. A method, comprising:
monitoring, with one or more first sensors disposed in a sensor device that is positioned at least partially within an ear canal of a user, physiological data of the user;
sending, with the one or more first sensors and to a computing system, data regarding the monitored physiological data of the user as obtained from within the ear canal of the user;
receiving, with the computing system, the data regarding the monitored physiological data of the user as obtained from within the ear canal of the user;
analyzing, with the computing system, the received data regarding the monitored physiological data of the user as obtained from within the ear canal of the user;
determining, with the computing system, at least one physiological state of the user, based at least in part on the analysis of the data regarding the monitored physiological data of the user as obtained from within the ear canal of the user;
sending, with the computing system and to a user device, the determined at least one physiological state of the user; and
displaying, on a display screen of the user device, the determined at least one physiological state of the user.

2. The method of claim 1, wherein the one or more first sensors are encapsulated within a sensor device, wherein the sensor device comprises an ear-plug-shaped housing configured to fit within the ear canal of the user.

3. The method of claim 1, further comprising:
monitoring, with one or more second sensors disposed in a second sensor device that is positioned at least partially within a second ear canal of the user, second physiological data of the user;
sending, with the one or more second sensors and to a computing system, data regarding the monitored physiological data of the user as obtained from within the second ear canal of the user;
wherein the second sensor device is positioned at least partially within a first ear canal of the user.

4. The method of claim 1, wherein the monitored physiologic data includes pulsatile waveform data, wherein monitoring the physiological data of the user further comprises monitoring motion data from within the ear canal of the user, the method further comprising:
mitigating motion artifacts in the pulsatile waveform data based, at least in part, on the monitored motion data.

5. The method of claim 1, wherein monitoring the physiological data of the user comprises monitoring a thermal reading from within the ear canal of the user, wherein determining the at least one physiological state of the user comprises determining, with the computing system, a body temperature of the user, based at least in part on the monitored thermal reading.

6. The method of claim 1, wherein monitoring the physiological data of the user comprises monitoring a pulsatile waveform that is detected in the ear canal of the user, wherein determining the at least one physiological state of the user comprises calculating, with the computing system, at least one of a compensatory reserve index (“CRI”) or a heart rate (“HR”) of the user based at least in part on the monitored pulsatile waveform.

7. The method of claim 1, wherein displaying the determined at least one physiological state of the user comprises at least one of:
displaying, on the display screen of the user device, an indication that the sensor device has been properly placed within the ear canal to sufficiently capture sensor readings;
displaying, on the display screen of the user device, an indication that the sensor device has not been properly placed within the ear canal to sufficiently capture sensor readings and a set of instructions to properly insert the sensor device within the ear canal after removal of the sensor device from the ear canal;
displaying, on the display screen of the user device, a body temperature of the user as measured by the one or more first sensors from within the ear canal of the user;
displaying, on the display screen of the user device, a compensatory reserve index (“CRI”) as calculated based at least in part on pulsatile waveform readings as obtained by the one or more first sensors from within the ear canal of the user;
displaying, on the display screen of the user device, a heart rate (“HR”) of the user as measured by the one or more first sensors from within the ear canal of the user;
displaying, on the display screen of the user device, at least one notification indicating that one or more of CRI, HR, or body temperature readings are valid and complete; or
displaying, on the display screen of the user device, one or more error messages when at least one of an error occurs or no valid readings can be made.

8. The method of claim 1, further comprising:
audibly presenting, using one or more speakers of the user device, an indication that the sensor device has been properly placed within the ear canal to sufficiently capture sensor readings;
audibly presenting, using the one or more speakers of the user device, an indication that the sensor device has not been properly placed within the ear canal to sufficiently capture sensor readings and a set of instructions to properly insert the sensor device within the ear canal after removal of the sensor device from the ear canal;
audibly presenting, using the one or more speakers of the user device, a body temperature of the user as measured by the one or more first sensors from within the ear canal of the user;
audibly presenting, using the one or more speakers of the user device, a compensatory reserve index (“CRI”) as calculated based at least in part on pulsatile waveform readings as obtained by the one or more first sensors from within the ear canal of the user;
audibly presenting, using the one or more speakers of the user device, a heart rate (“HR”) of the user as measured by the one or more first sensors from within the ear canal of the user;
audibly presenting, using the one or more speakers of the user device, one or more beeps or tones indicating that one or more of CRI, HR, or body temperature readings are valid and complete; or
audibly presenting, using the one or more speakers of the user device, one or more error messages when at least one of an error occurs or no valid readings can be made.

9. An apparatus, comprising:
at least one processor; and
a non-transitory computer readable medium communicatively coupled to the at least one processor, the non-transitory computer readable medium having stored thereon computer software comprising a set of instructions that, when executed by the at least one processor, causes the apparatus to:
receive, from one or more first sensors disposed in a sensor device that is positioned at least partially within an ear canal of a user, data regarding monitored physiological data of the user as obtained from within the ear canal of the user;
analyze the received data regarding the monitored physiological data of the user as obtained from within the ear canal of the user;
determine at least one physiological state of the user, based at least in part on the analysis of the data regarding the monitored physiological data of the user as obtained from within the ear canal of the user;
send, to a user device, the determined at least one physiological state of the user; and
displaying, on a display screen of the user device, the determined at least one physiological state of the user.

10. The apparatus of claim 9, wherein the set of instructions is further executable by the processor to:
monitor, with one or more second sensors disposed in a second sensor device that is positioned at least partially within a second ear canal of the user, second physiological data of the user;
send, with the one or more second sensors and to a computing system, data regarding the monitored physiological data of the user as obtained from within the second ear canal of the user;
wherein the second sensor device is positioned at least partially within a first ear canal of the user.

11. The apparatus of claim 9, wherein the monitored physiologic data includes pulsatile waveform data, wherein monitoring the physiological data of the user further comprises monitoring motion data from within the ear canal of the user, wherein the first set of instructions is further executable by the processor to:
mitigate motion artifacts in the pulsatile waveform data based, at least in part, on the monitored motion data.

12. A system, comprising:
one or more first sensors, wherein the one or more first sensors are disposed in a sensor device that is positioned at least partially within an ear canal of a user, wherein the one or more first sensors are used to monitor physiological data of the user;
a computing system, comprising:
at least one first processor; and
a first non-transitory computer readable medium communicatively coupled to the at least one first processor, the first non-transitory computer readable medium having stored thereon computer software comprising a first set of instructions that, when executed by the at least one first processor, causes the computing system to:
receive, from the one or more first sensors, data regarding monitored physiological data of the user as obtained from within the ear canal of the user;
analyze the received data regarding the monitored physiological data of the user as obtained from within the ear canal of the user;
determine at least one physiological state of the user, based at least in part on the analysis of the data regarding the monitored physiological data of the user as obtained from within the ear canal of the user;
send, to a user device, the determined at least one physiological state of the user; and
displaying, on a display screen of the user device, the determined at least one physiological state of the user.

13. The system of claim 12, wherein the one or more first sensors comprise at least one of one or more thermal sensors, one or more optical sensors, or one or more heart rate sensors.

14. The system of claim 12, wherein the one or more first sensors are encapsulated within a sensor device, wherein the sensor device comprises an ear-plug-shaped housing configured to fit within the ear canal of the user.

15. The system of claim 12, wherein the first set of instructions is further executable by the processor to:
monitor, with one or more second sensors disposed in a second sensor device that is positioned at least partially within a second ear canal of the user, second physiological data of the user;
send, with the one or more second sensors and to a computing system, data regarding the monitored physiological data of the user as obtained from within the second ear canal of the user;
wherein the second sensor device is positioned at least partially within a first ear canal of the user.

16. The system of claim 12, wherein the monitored physiologic data includes pulsatile waveform data, wherein monitoring the physiological data of the user further comprises monitoring motion data from within the ear canal of the user, wherein the first set of instructions is further executable by the processor to:
mitigate motion artifacts in the pulsatile waveform data based, at least in part, on the monitored motion data.

17. The system of claim 12, wherein monitoring the physiological data of the user comprises monitoring a thermal reading from within the ear canal of the user, wherein determining the at least one physiological state of the user comprises determining a body temperature of the user, based at least in part on the monitored thermal reading.

18. The system of claim 12 wherein monitoring the physiological data of the user comprises monitoring a pulsatile waveform that is detected in the ear canal of the user, wherein determining the at least one physiological state of the user comprises calculating at least one of a compensatory reserve index (“CRI”) or a heart rate (“HR”) of the user based at least in part on the monitored pulsatile waveform.

19. The system of claim 12, wherein displaying the determined at least one physiological state of the user comprises at least one of:
displaying, on the display screen of the user device, an indication that the sensor device has been properly placed within the ear canal to sufficiently capture sensor readings;
displaying, on the display screen of the user device, an indication that the sensor device has not been properly placed within the ear canal to sufficiently capture sensor readings and a set of instructions to properly insert the sensor device within the ear canal after removal of the sensor device from the ear canal;
displaying, on the display screen of the user device, a body temperature of the user as measured by the one or more first sensors from within the ear canal of the user;
displaying, on the display screen of the user device, a compensatory reserve index (“CRI”) as calculated based at least in part on pulsatile waveform readings as obtained by the one or more first sensors from within the ear canal of the user;
displaying, on the display screen of the user device, a heart rate (“HR”) of the user as measured by the one or more first sensors from within the ear canal of the user;
displaying, on the display screen of the user device, at least one notification indicating that one or more of CRI, HR, or body temperature readings are valid and complete; or
displaying, on the display screen of the user device, one or more error messages when at least one of an error occurs or no valid readings can be made.

20. The system of claim 12, wherein the first set of instructions, when executed by the at least one first processor, further causes the computing system to perform at least one of:
audibly presenting, using one or more speakers of the user device, an indication that the sensor device has been properly placed within the ear canal to sufficiently capture sensor readings;
audibly presenting, using the one or more speakers of the user device, an indication that the sensor device has not been properly placed within the ear canal to sufficiently capture sensor readings and a set of instructions to properly insert the sensor device within the ear canal after removal of the sensor device from the ear canal;
audibly presenting, using the one or more speakers of the user device, a body temperature of the user as measured by the one or more first sensors from within the ear canal of the user;
audibly presenting, using the one or more speakers of the user device, a compensatory reserve index (“CRI”) as calculated based at least in part on pulsatile waveform readings as obtained by the one or more first sensors from within the ear canal of the user;
audibly presenting, using the one or more speakers of the user device, a heart rate (“HR”) of the user as measured by the one or more first sensors from within the ear canal of the user;
audibly presenting, using the one or more speakers of the user device, one or more beeps or tones indicating that one or more of CRI, HR, or body temperature readings are valid and complete; or
audibly presenting, using the one or more speakers of the user device, one or more error messages when at least one of an error occurs or no valid readings can be made.
